SEQUENCE LISTING (4 sequences)

SEQ ID NO: 1 - DNA, 20 bp, synthetic construct
  source 1..20: mol_type = other DNA
tcaccaacggtggcatctac

SEQ ID NO: 2 - DNA, 20 bp, synthetic construct
  source 1..20: mol_type = other DNA
tcatctgctggatgtcgtcg

SEQ ID NO: 3 - DNA, 20 bp, synthetic construct
  source 1..20: mol_type = other DNA
tcaccaacggtggcatctac

SEQ ID NO: 4 - DNA, 20 bp, synthetic construct
  source 1..20: mol_type = other DNA
cactccgatggcttcgatga

(19) United States
(12) Patent Application Publication
(10) Pub. No.: US 2026/0158026 A1
(43) Pub. Date: Jun. 11, 2026

(54) METHODS OF TREATING CANCER AND COMPOSITIONS FOR THE SAME

(71) Applicant: MARSAL THERAPEUTICS, INC., Austin, TX (US)

(72) Inventors: Satish Srinivas Kitambi, Secunderabad (IN); Gayathri Chandrasekar-Janebjer, Secunderabad (IN)

(21) Appl. No.: 19/115,688

(22) PCT Filed: Sep. 25, 2023
(86) PCT No.: PCT/US2023/074989

§ 371 Date: Mar. 26, 2025

Related U.S. Application Data
(60) Provisional application No. 63/382,653, filed on Nov. 7, 2022.

(30) Foreign Application Priority Data
Sep. 26, 2022 (IN) 202211055079

(51) Int. Cl. A61K 31/495; A61K 31/357; A61K 31/4166; A61K 31/427; A61K 31/55; A61K 31/7076; A61P 35/00

(52) U.S. Cl. CPC A61K 31/495; A61K 31/357; A61K 31/4166; A61K 31/427; A61K 31/55; A61K 31/7076; A61P 35/00

(57) ABSTRACT

The present invention provides a method of treating cancer in a subject in need thereof, comprising administering to the subject Compound 1 (I) or a pharmaceutically acceptable salt thereof, or a pharmaceutical composition comprising Compound 1 or a pharmaceutically acceptable salt thereof. Also provided are pharmaceutical compositions for treating cancer.

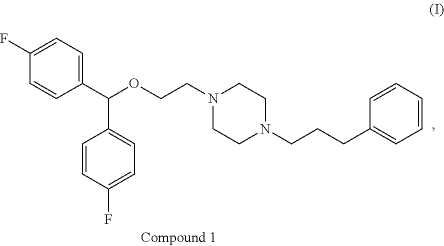 [SMILES: Fc1ccc(C(OCCN2CCN(CCCc3ccccc3)CC2)c2ccc(F)cc2)cc1]

### CROSS REFERENCE TO RELATED APPLICATION

This application is a 35 U.S.C. § 371 United States national phase application of PCT Application No. PCT/US2023/074989, filed Sep. 25, 2023, which claims the benefit of Indian Patent Application number 202211055079, filed Sep. 26, 2022, and U.S. provisional application No. 63/382,653, filed Nov. 7, 2022. The entire contents of the aforementioned applications are incorporated herein by reference in their entireties.

### SEQUENCE LISTING

This application incorporates by reference in its entirety the Sequence Listing XML entitled “272050-564890” (4,562 bytes), which was created on Feb. 25, 2026, and filed electronically herewith.

### FIELD OF THE INVENTION

The present invention relates to compounds and methods of treating cancer in a subject in need thereof.

### BACKGROUND

Cancer remains one of the most deadly threats to human health. Certain cancers can metastasize and grow rapidly in an uncontrolled manner, making timely detection and treatment extremely difficult. In the United States, cancer affects nearly 1.9 million new patients each year and is the second leading cause of death after heart disease. While numerous anticancer agents are approved for use therapeutically, very few have activity against a broad spectrum of cancers. This is because there can be very significant differences among individual cancers. Additionally, most anticancer agents are believed to treat cancer via multiple mechanisms of action, further diminishing the number of agents that are broadly efficacious against a broad spectrum of cancers. Most current efforts focus on cancer treatment, with less emphasis placed on patient quality of life during cancer treatment and management of end of life care, or palliative treatment for glioblastoma multiforme patients whose condition is terminal.

Gliomas are a class of primary tumors that originate in the brain and/or spine. The most common type of gliomas are astrocytomas. Astrocytomas are graded on a scale from I to IV based on how normal or abnormal the cells appear: Grade I—pilocytic astrocytoma; Grade II—diffuse astrocytoma; Grade III—anaplastic astrocytoma; and Grade IV—glioblastoma, also known as glioblastoma multiforme (GBM). Low-grade astrocytomas are usually localized and grow slowly. High-grade astrocytomas grow at a rapid pace and require a different course of treatment.

GBM accounts for about 48% of all primary malignant brain tumors, and usually results in death within 12-15 months of diagnosis. GBM has a five-year survival rate of 3-7%.

Currently, there is no known method of preventing GBM. Furthermore, treatment of GBM can be extremely difficult due to a variety of factors, including the brain's limited capacity for self-repair, difficulty in drugs passing the blood-brain barrier, potential of damage to the brain by use of conventional therapies (such as aggressive chemotherapy, radiation, and/or surgery), and difficulty in surgically removing all cancerous cells due to infiltration of the tumor throughout the brain. Due to these aforementioned challenges in treating GBM, most care for GBM patients involves palliative care, care to help slow the spread, and other measures aimed at temporarily improving survival and/or lifestyle of GBM patients.

Accordingly, there remains a need for compounds and methods for treating cancer generally, and GBM specifically. The compound, pharmaceutically acceptable salts thereof, pharmaceutical compositions, and methods described herein are directed toward this end.

### SUMMARY OF THE INVENTION

The present invention provides a method of treating cancer in a subject in need thereof, comprising administering to the subject Compound 1

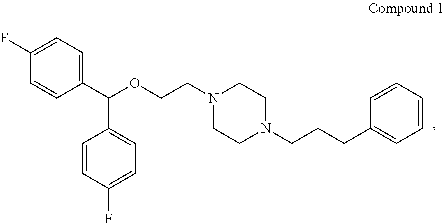 [SMILES: Fc1ccc(C(OCCN2CCN(CCCc3ccccc3)CC2)c2ccc(F)cc2)cc1]

or a pharmaceutically acceptable salt thereof.

In one aspect, the invention provides a method of treating cancer in a subject in need thereof, comprising administering to the subject a therapeutically effective amount of Compound 1

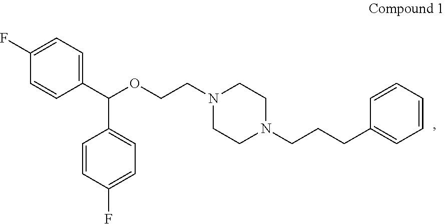 [SMILES: Fc1ccc(C(OCCN2CCN(CCCc3ccccc3)CC2)c2ccc(F)cc2)cc1]

or a pharmaceutically acceptable salt thereof, wherein the cancer is selected from brain cancer, breast cancer, pancreatic cancer, lung cancer, or any combination thereof.

In some implementations, Compound 1, or the pharmaceutically acceptable salt thereof, is a hydrochloride salt of Compound 1. And, in some implementations, Compound 1, or the pharmaceutically acceptable salt thereof, is a dihydrochloride salt of Compound 1.

In some implementations, the cancer is brain cancer.

In some implementations, Compound 1, or the pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in cancer cells. In other implementations, Compound 1, or the pharmaceutically acceptable salt thereof, modulates activity of BMPR1A, UTP23, ARHGAP33, ITSN1, or any combination thereof in cancer cells.

In some implementations, Compound 1, or the pharmaceutically acceptable salt thereof, is administered as a pharmaceutical composition comprising a pharmaceutically acceptable carrier, excipient, or diluent. In some implementations, Compound 1, or the pharmaceutically acceptable salt thereof, is administered at least once per day (e.g., once per day, at least twice per day, twice per day, or three times per day).

In some implementations, the method further comprises administering to the subject an additional therapeutic agent. In some implementations, the additional therapeutic agent is an anti-cancer agent. In some implementations, the anti-cancer agent is selected from temozolomide, ixabepilone, cladribine, enzalutamide, omacetaxine mepesuccinate, epothilones, erubulin, latrunculin, a pharmaceutically acceptable salt of any of these anti-cancer agents, or any combination thereof. In some implementations, Compound 1, or the pharmaceutically acceptable salt thereof, and the additional therapeutic agent are administered concurrently. In some implementations, Compound 1, or the pharmaceutically acceptable salt thereof, and the additional therapeutic agent are administered sequentially. And, in some implementations, the method further comprises administering to the subject a radiation therapy.

In some implementations, Compound 1, or the pharmaceutically acceptable salt thereof, is administered in a dosage amount of from about 1 mg to about 1,000 mg. In other implementations, Compound 1, or the pharmaceutically acceptable salt thereof, is administered in a dosage amount of from about 25 mg to about 750 mg. In other implementations, Compound 1, or the pharmaceutically acceptable salt thereof, is administered in a dosage amount of from about 50 mg to about 500 mg. And, in some embodiments, Compound 1, or the pharmaceutically acceptable salt thereof, is administered in a dosage amount of from about 75 mg to about 300 mg.

In some implementations, Compound 1, or the pharmaceutically acceptable salt thereof, is administered chronically. In some implementations, Compound 1, or the pharmaceutically acceptable salt thereof, is administered orally, subcutaneously, intramuscularly, intravenously, intracranially, intrathecally, or intranasally. In some implementations, Compound 1, or the pharmaceutically acceptable salt thereof, is administered orally. In some implementations, Compound 1, or the pharmaceutically acceptable salt thereof, is administered as a tablet, a capsule, or an oral suspension. In some implementations, Compound 1, or the pharmaceutically acceptable salt thereof, is administered at least once a day from onset of treatment for a duration of at least about one year.

In some implementations, the subject experiences a reduced loss in bodyweight following treatment onset as compared to a subject not administered Compound 1 or a pharmaceutically acceptable salt thereof. In some implementations, the subject experiences a vacuolization of mitochondria in cancer cells following treatment onset. And, in some implementations, the subject is a human.

Another aspect of the present invention provides a method of treating glioma in a subject in need thereof, comprising administering to the subject a therapeutically effective amount of Compound 1

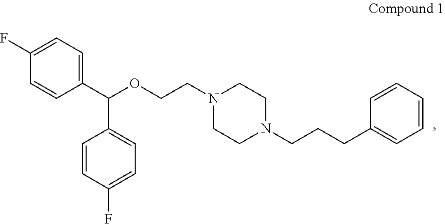 [SMILES: Fc1ccc(C(OCCN2CCN(CCCc3ccccc3)CC2)c2ccc(F)cc2)cc1]

or a pharmaceutically acceptable salt thereof.

In some implementations, the Compound 1, or the pharmaceutically acceptable salt thereof, is a hydrochloride salt of Compound 1. And, in some implementations, the hydrochloride salt is a dihydrochloride salt of Compound 1.

In some implementations, the glioma is selected from the group consisting of an astrocytoma, an ependymoma, an oligodendroglioma, a brainstem glioma, an optic nerve glioma, and a mixed glioma. In some implementations, the glioma is an astrocytoma. And, in some implementations, the astrocytoma is glioblastoma multiforme (GBM).

In some implementations, Compound 1, or the pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in glioma cells. In other implementations, Compound 1, or the pharmaceutically acceptable salt thereof, modulates activity of BMPR1A, UTP23, ARHGAP33, ITSN1, or any combination thereof in glioma cells.

In some implementations, Compound 1 or the pharmaceutically acceptable salt thereof, is administered as a pharmaceutical composition additionally comprising a pharmaceutically acceptable carrier, excipient, or diluent. In some implementations, Compound 1, or the pharmaceutically acceptable salt thereof, is administered at least once per day (e.g., once per day, at least twice per day, twice per day, or three times per day).

In some implementations, the method further comprises administering to the subject an additional therapeutic agent. In some implementations, the additional therapeutic agent is an anti-cancer agent. In some implementations, the anti-cancer agent is selected from temozolomide, ixabepilone, cladribine, enzalutamide, omacetaxine mepesuccinate, epothilones, erubulin, latrunculin, a pharmaceutically acceptable salt of any of these anti-cancer agents, or any combination thereof. In some implementations, Compound 1, or the pharmaceutically acceptable salt thereof, and the additional therapeutic agent are administered concurrently. In some implementations, Compound 1, or the pharmaceutically acceptable salt thereof, and the additional therapeutic agent are administered sequentially. And, in some implementations, the method further comprises administering to the subject a radiation therapy.

In some implementations, Compound 1, or the pharmaceutically acceptable salt thereof, is administered in a dosage amount of from about 1 mg to about 1,000 mg. In other implementations, Compound 1, or the pharmaceutically acceptable salt thereof, is administered in a dosage amount of from about 25 mg to about 750 mg. In other implementations, Compound 1, or the pharmaceutically acceptable salt thereof, is administered in a dosage amount of from about 50 mg to about 500 mg. And, in some embodiments, Compound 1, or the pharmaceutically acceptable salt thereof, is administered in a dosage amount of from about 75 mg to about 300 mg.

In some implementations, Compound 1, or the pharmaceutically acceptable salt thereof, is administered chronically. In some implementations, Compound 1, or the pharmaceutically acceptable salt thereof, is administered orally, subcutaneously, intramuscularly, intravenously, intracranially, intrathecally, or intranasally. In some implementations, Compound 1, or the pharmaceutically acceptable salt thereof, is administered orally. In some implementations, Compound 1, or the pharmaceutically acceptable salt thereof, is administered as a tablet, capsule, or oral suspension. In some implementations, Compound 1, or the pharmaceutically acceptable salt thereof, is administered at least once a day from onset of treatment for a duration of at least about one year.

In some implementations, the subject experiences a reduced loss in bodyweight following treatment onset as compared to a subject not administered Compound 1, or a pharmaceutically acceptable salt thereof. In some implementations, the subject experiences a vacuolization of mitochondria in glioma cells following treatment onset. And, in some implementations, the subject is a human.

Another aspect of the present invention provides a method of inducing vacuolization of mitochondria in glioblastoma multiforme (GBM) cells of a subject, comprising administering to the subject a therapeutically effective amount of Compound 1

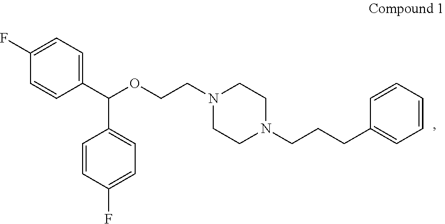 [SMILES: Fc1ccc(C(OCCN2CCN(CCCc3ccccc3)CC2)c2ccc(F)cc2)cc1]

or a pharmaceutically acceptable salt thereof.

In some implementations, Compound 1, or the pharmaceutically acceptable salt thereof, is a hydrochloride salt of Compound 1. And, in some implementations, the hydrochloride salt is a dihydrochloride salt of Compound 1.

In some implementations, Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in GBM cells. In other implementations, Compound 1, or the pharmaceutically acceptable salt thereof, modulates BMPR1A, UTP23, ARHGAP33, ITSN1, or any combination thereof in GBM cells.

In some implementations, Compound 1, or the pharmaceutically acceptable salt thereof, is administered as a pharmaceutical composition additionally comprising a pharmaceutically acceptable carrier, excipient, or diluent. In some implementations, Compound 1, or the pharmaceutically acceptable salt thereof, is administered at least once per day.

In some implementations, Compound 1, or the pharmaceutically acceptable salt thereof, is administered in a dosage amount of from about 1 mg to about 1,000 mg. In other implementations, Compound 1, or the pharmaceutically acceptable salt thereof, is administered in a dosage amount of from about 25 mg to about 750 mg. In other implementations, Compound 1, or the pharmaceutically acceptable salt thereof, is administered in a dosage amount of from about 50 mg to about 500 mg. And, in some embodiments, Compound 1, or the pharmaceutically acceptable salt thereof, is administered in a dosage amount of from about 75 mg to about 300 mg.

In some implementations, Compound 1, or the pharmaceutically acceptable salt thereof, is administered chronically. In some implementations, Compound 1, or the pharmaceutically acceptable salt thereof, is administered orally, subcutaneously, intramuscularly, intravenously, intracranially, intrathecally, or intranasally. In some implementations, Compound 1, or the pharmaceutically acceptable salt thereof, is administered orally. In some implementations, Compound 1, or the pharmaceutically acceptable salt thereof, is administered as a tablet, capsule, or oral suspension. In some implementations, Compound 1, or the pharmaceutically acceptable salt thereof, is administered at least once a day from onset of treatment for a duration of at least about one year. And, in some implementations, the subject is a human.

Another aspect of the present invention provides a method of depolarizing mitochondria in glioblastoma multiforme (GBM) cells of a subject, comprising administering to the subject a therapeutically effective amount of Compound 1

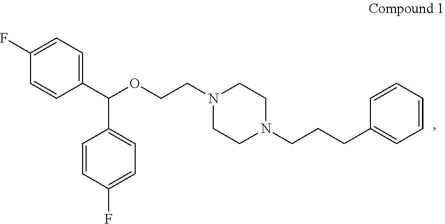 [SMILES: Fc1ccc(C(OCCN2CCN(CCCc3ccccc3)CC2)c2ccc(F)cc2)cc1]

or a pharmaceutically acceptable salt thereof.

In some implementations, Compound 1, or the pharmaceutically acceptable salt thereof, is a hydrochloride salt of Compound 1. And, in some implementations, the hydrochloride salt is a dihydrochloride salt of Compound 1.

In some implementations, Compound 1, or the pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in GBM cells. In some implementations, Compound 1, or the pharmaceutically acceptable salt thereof, modulates BMPR1A, UTP23, ARHGAP33, ITSN1, or any combination thereof in GBM cells.

In some implementations, Compound 1, or the pharmaceutically acceptable salt thereof, is administered as a pharmaceutical composition additionally comprising a pharmaceutically acceptable carrier, excipient, or diluent. In some implementations, Compound 1, or the pharmaceutically acceptable salt thereof, is administered at least once per day.

In some implementations, Compound 1, or the pharmaceutically acceptable salt thereof, is administered in a dosage amount of from about 1 mg to about 1,000 mg. In other implementations, Compound 1, or the pharmaceutically acceptable salt thereof, is administered in a dosage amount of from about 25 mg to about 750 mg. In other implementations, Compound 1, or the pharmaceutically acceptable salt thereof, is administered in a dosage amount of from about 50 mg to about 500 mg. And, in some embodiments, Compound 1, or the pharmaceutically acceptable salt thereof, is administered in a dosage amount of from about 75 mg to about 300 mg.

In some implementations, Compound 1, or the pharmaceutically acceptable salt thereof, is administered chronically. In some implementations, Compound 1, or the pharmaceutically acceptable salt thereof, is administered orally, subcutaneously, intramuscularly, intravenously, intracranially, intrathecally, or intranasally. In some implementations, Compound 1, or the pharmaceutically acceptable salt thereof, is administered orally. In some implementations, Compound 1, or the pharmaceutically acceptable salt thereof, is administered as a tablet, capsule, or oral suspension. In some implementations, Compound 1, or the pharmaceutically acceptable salt thereof, is administered at least once a day from onset of treatment for a duration of at least about one year. In some implementations, the subject experiences a vacuolization of mitochondria in GBM cells following treatment onset. And, in some implementations, the subject is a human.

Another aspect of the present invention provides a pharmaceutical composition for treating glioma in a subject, wherein the pharmaceutical composition comprises Compound 1

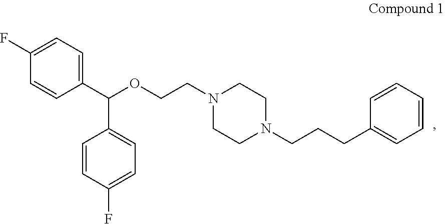 [SMILES: Fc1ccc(C(OCCN2CCN(CCCc3ccccc3)CC2)c2ccc(F)cc2)cc1]

or a pharmaceutically acceptable salt thereof.

In some embodiments, Compound 1, or the pharmaceutically acceptable salt thereof, is a hydrochloride salt of Compound 1. And, in some embodiments, the hydrochloride salt is a dihydrochloride salt of Compound 1.

In some embodiments, the pharmaceutical composition is a nasal spray having a viscosity of from about 100 cP to about 2,500 cP. And, in some embodiments, the pharmaceutical composition comprises a tablet, capsule, or oral suspension.

### BRIEF DESCRIPTION OF THE DRAWINGS

The figures below are provided by way of example and are not intended to limit the scope of the claimed invention.

FIG. 1A is a plot of cell viability of patient-derived cell lines treated with various classes of compounds according to Example 1.

FIG. 1B is a graph of cell viability of patient-derived GBM cells (GBM) and fibroblasts (HF) treated with Compound 1 (CMPD1) according to Example 3.

FIG. 1C is a graph of cell viability of patient-derived GBM cells and fibroblasts across multiple passages, after 4 days post-treatment with Compound 1, according to Example 3.

FIG. 1D is a graph of cell viability of patient-derived fibroblasts across multiple passages, after four days post treatment with Compound 1, according to Example 3.

FIG. 2 is a graph of cell viability of patient-derived GBM cells (isolated from an additional five patients) after treatment with Compound 1 according to Example 3.

FIG. 3A is bar graphs of cell viability of patient-derived GBM cells treated with DMSO or Compound 1 (0.1 μM or 5 μM) at day 4, day 8, day 14, and day 21 according to Example 4.

FIG. 3B is bar graphs of cell viability of patient-derived fibroblasts (control) treated with DMSO or Compound 1 (0.1 μM) at 4 day and at day 21 according to Example 4.

FIG. 4A is a graph of cell viability of patient-derived GBM cells post knockdown with scrambled (control) siRNA or DAT specific siRNA treated with Compound 1 according to Example 5.

FIG. 4B is a bar graph of relative DAT expression in patient-derived GBM cells post knockdown with scrambled (control) siRNA or DAT specific siRNA according to Example 5.

FIG. 4C is a graph of cytotoxicity/apoptosis of patient-derived GBM cells with apoptotic marker Caspase 3, post-treatment with DMSO (Control), Compound 1 (CMPD1) or with staurosporin (positive control for apoptosis) according to Example 5.

FIG. 4D is an image for quantification of apoptotic cells represented as percentage of total cells that were analyzed from FIG. 4C according to Example 5.

FIG. 5A is plots of membrane potential for GMB cells treated with DMSO (negative control), CCCP (positive control), and Compound 1 (CMPD1) according to Example 6.

FIG. 5B is a bar graph of JC1 staining for mitochondrial depolarization in GMB cells treated with DMSO (negative control), CCCP (positive control), or Compound 1 (CMPD1) at 24 hrs, 48 hrs, and 72 hrs according to Example 6.

FIG. 6A is TEM images of mitochondria of GBM cells treated with Compound 1 (CMPD1) (2.5 μM, 5 μM, or 10 μM) or DMSO according to Example 7.

FIG. 6B is a bar graph of mitochondria vacuolization in GMB cells treated with DMSO or Compound 1 (2.5 μM, 5 μM, or 10 μM) at 3 hrs, 6 hrs, 12 hrs, 24 hrs, and 48 hrs according to Example 7.

FIG. 7A is a step diagram of the CRISPR-Cas9 whole genome screening study to identify potential targets of Compound 1 in GBM cells according to Example 8.

FIG. 7B is a plot of the CRISPR-Cas9 whole genome screening study identifying potential targets of Compound 1 in GBM cells according to Example 8.

FIG. 7C is a graph of cell viability after treatment with Compound 1 for certain targets identified in FIG. 7B in patient-derived glioma cells followed by measurement of IC₅₀ post-treatment with serial dilution of Compound 1 according to Example 8.

FIG. 8A is photographs of electrophoresis gels showing expression of certain targets identified in FIG. 7B post-treatment with DMSO (control), Compound 1 (CMPD1), Biotin (control) and Biotin labeled Compound 1 (CMPD1-Biotin) according to Example 8.

FIG. 8B is a photograph of electrophoresis gels showing the results of a pull-down assay with biotin labeled Compound 1 according to Example 8.

FIG. 9 is photographs showing GBM tumor size in a mouse treated with Compound 1 and an untreated mouse according to Example 9.

FIG. 10 is a graph comparing average body weight of mice treated with Compound 1 and untreated mice according to Example 9.

FIG. 11 is a graph comparing survival rate of mice treated with Compound 1 and untreated mice according to Example 9.

FIG. 12A is a graph of cell viability for patient-derived GBM cells treated with temozolomide (TMZ) and a combination of TMZ and Compound 1 according to Example 10.

FIG. 12B is a bar graph of cell viability for patient-derived GBM cells treated with Compound 1 (GK09) and/or compounds approved for use as oncology therapeutics by the FDA according to Example 10.

FIG. 12C is a graph of cell viability for patient-derived GBM cells treated with Compound 1 (GK09) and/or compounds approved for use as oncology therapeutics by the FDA including the measured IC₅₀ values according to Example 10.

### DETAILED DESCRIPTION

The present invention provides compounds and methods of treating cancer in a subject in need thereof.

As used herein, the following definitions shall apply unless otherwise indicated.

### I. DEFINITIONS

As used herein, the term “modulates” refers to the inhibition or potentiation of BMPR1A, UTP23, ARHGAP33, ITSN1, CARD1, ETV2L, HMGN5, LYSMD4, PLA2G7, TAF11, TDRD5, UTP23, UGP2, WT1P, and/or ZNF75A function. A “modulator” (e.g., a compound or pharmaceutically acceptable salt thereof that modulates BMPR1A, UTP23, ARHGAP33, ITSN1, CARD1, ETV2L, HMGN5, LYSMD4, PLA2G7, TAF11, TDRD5, UTP23, UGP2, WT1P, and/or ZNF75A function) may be, for example, an agonist, partial agonist, antagonist, or partial antagonist of BMPR1A, UTP23, ARHGAP33, ITSN1, CARD1, ETV2L, HMGN5, LYSMD4, PLA2G7, TAF11, TDRD5, UTP23, UGP2, WT1P, and/or ZNF75A.

As used herein, the term “about”, when referring to a numerical value or range of values, allows for a degree of variability in the value or range or values, for example, within 10%, or within 5% of a stated value or of a stated limit of a range.

As used herein, the term “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, the term “pharmaceutically acceptable salt” refers to a salt of a compound of the invention that is pharmaceutically acceptable and that possesses the desired pharmacological activity of the parent compound. In particular, such salts are non-toxic, and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counterion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

As used herein, the term “prodrug” is intended to encompass therapeutically inactive compounds that, under physiological conditions, are converted into the therapeutically active agents of the present invention. One method for making a prodrug is to design selected moieties that are hydrolyzed or cleaved at a targeted in vivo site of action under physiological conditions to reveal the desired molecule which then produces its therapeutic effect. In certain embodiments, the prodrug is converted by an enzymatic activity of the subject.

In an alternate embodiment, the present invention provides prodrugs of compounds described herein (e.g., Compound 1).

As used herein, the term “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a mammal. In other embodiments, the subject is a human.

As used herein, and unless otherwise specified, the terms “treat,” “treating,” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

In general, the “effective amount” of a compound refers to an amount sufficient to elicit the desired biological response, e.g., to treat cancer (e.g., glioblastoma multiforme (GBM)). As will be appreciated by those of ordinary skill in the art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, the term “treatment onset” and “onset of treatment” are used interchangeably to refer to the day in which a treatment (e.g., administration of Compound 1 or a pharmaceutically acceptable salt thereof) begins. For example, the day of treatment onset is the day in which a chemotherapy is first administered. For dosage regimes comprising the administration of 2 or more therapies, treatment onset is the day in which Compound 1 or a pharmaceutically acceptable salt thereof is first administered.

As used herein, a “halogen” or “halo” group refers to fluorine, chlorine, bromine or iodine.

Unless otherwise stated, structures depicted herein also are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

Compounds disclosed herein may be “isomerically pure” compounds. As used herein, the term “isomerically pure” refers to an isomeric form of a compound that is substantially free from other isomeric forms of the compound (e.g., substantially free from other stereoisomers (e.g., enantiomers, diastereomers, geometric (or conformational) isomers, etc.), constitutional isomers, isotopomers, etc.). For example, an “isomerically pure” compound having at least one asymmetric center of a particular configuration (i.e., R or S configuration) is substantially free from other isomeric forms of the compound having a different configuration at the at least one asymmetric center. An “isomerically pure” compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight, or more than 99.9% by weight, of a single isomer of the compound based on the total weight of all isomers of the compound that are present.

Chemical structures and nomenclature are derived from ChemDraw, version 11.0.1, Cambridge, MA.

### II. COMPOUNDS OF THE PRESENT INVENTION

The present invention provides compounds useful for treating cancer.

### A. Compounds of the Present Invention

One aspect of the present invention provides Compound 1

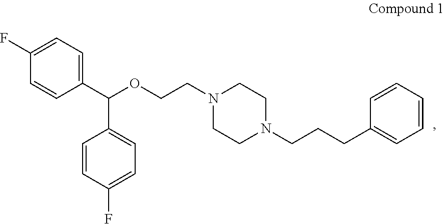 [SMILES: Fc1ccc(C(OCCN2CCN(CCCc3ccccc3)CC2)c2ccc(F)cc2)cc1]

or a pharmaceutically acceptable salt thereof.

In some embodiments, the present invention provides a pharmaceutically acceptable salt of Compound 1. For example, the pharmaceutically acceptable salt is a hydrochloride or dihydrochloride salt of Compound 1. In other examples, the pharmaceutically acceptable salt is a dihydrochloride salt of Compound 1.

### B. Pharmaceutical Compositions/Formulations

The compounds described herein can be formulated into pharmaceutical compositions that further comprise a pharmaceutically acceptable carrier, diluent, adjuvant or vehicle. In one embodiment, the present invention provides a pharmaceutical composition comprising a compound of the invention described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. In some embodiments, the present invention provides a pharmaceutical composition comprising a compound of the invention described above, or a pharmaceutically acceptable salt thereof, and a pharmaceutically acceptable carrier, excipient, or diluent. Pharmaceutically acceptable carriers include, for example, pharmaceutical diluents, excipients or carriers suitably selected with respect to the intended form of administration, and consistent with conventional pharmaceutical practices.

According to another embodiment, the invention provides a composition comprising a compound of this invention or a pharmaceutically acceptable salt thereof and a pharmaceutically acceptable carrier, excipient, or diluent. Pharmaceutical compositions of this invention comprise a therapeutically effective amount of Compound 1, or a pharmaceutically acceptable salt thereof.

It also will be appreciated that certain of the compounds of the present invention can exist in free form for treatment, or where appropriate, as a pharmaceutically acceptable derivative (e.g., a salt) thereof. According to the present invention, a pharmaceutically acceptable derivative includes, but is not limited to, pharmaceutically acceptable prodrugs, salts, esters, salts of such esters, or any other adduct or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A pharmaceutically acceptable carrier may contain inert ingredients that do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents. Preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid may be used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations also are prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, or preferentially, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds also can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also may comprise, as is normal practice, additional substances other than inert diluents, e.g., tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops also are contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers also can be used to increase the flux of the compound across the skin. The rate can be controlled by either providing a rate controlling membrane or by dispersing the compound in a polymer matrix or gel.

The compounds of the invention preferably are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Compounds and/or compositions of the invention can be delivered in a controlled release system. In one embodiment, a pump can be used to facilitate controlled release of the compounds and/or compositions of the invention (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, compositions of the invention can comprise polymeric materials to provide sustained, intermediate, pulsatile, or alternate release (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351(1989); Howard et al., J. Neurosurg. 71:105 (1989); Remington's Pharmaceutical Sciences, 18th Ed., (Mack Publishing Company, Easton, Pa., 1990)). Other controlled-release systems discussed in the review by Langer (Science 249:1527-1533 (1990)) can be used.

The amount of the compounds of the present invention that may be combined with the carrier materials to produce a composition in a single dosage form will vary depending upon the host treated, the particular mode of administration, and other factors. Preferably, the compositions should be formulated so that a dosage of between 0.01-100 mg/kg body weight/day of the inhibitor can be administered to a patient receiving these compositions.

Depending upon the particular condition, or disease, to be treated or prevented, additional therapeutic agents, which are normally administered to treat or prevent that condition, also may be present in the compositions of this invention. As used herein, additional therapeutic agents that are normally administered to treat or prevent a particular disease, or condition, are known as “appropriate for the disease, or condition, being treated.”

In one aspect, the invention provides a pharmaceutical composition for treating glioma in a subject, wherein the pharmaceutical composition comprises Compound 1

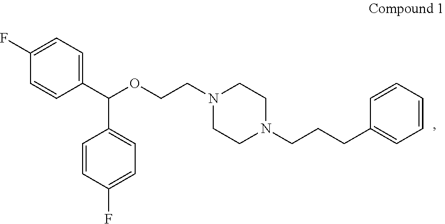 [SMILES: Fc1ccc(C(OCCN2CCN(CCCc3ccccc3)CC2)c2ccc(F)cc2)cc1]

or a pharmaceutically acceptable salt thereof.

In some embodiments, Compound 1, or a pharmaceutically acceptable salt thereof, is a hydrochloride salt. And, in some embodiments, the hydrochloride salt is a dihydrochloride salt.

In some embodiments, the pharmaceutical composition is a nasal spray having a viscosity of from about 100 cP to about 2,500 cP. In some embodiments, the nasal spray has a viscosity of at least about 100 cP, at least about 250 cP, at least about 500 cP, at least about 750 cP, at least about 1000 cP, at least about 1250 cP, at least about 1500 cP, at least about 1750 cP, at least about 2000 cP, at least about 2250 cP, or at least about 2500 cP, and/or no more than about 100 cP, no more than about 250 cP, no more than about 500 cP, no more than about 750 cP, no more than about 1000 cP, no more than about 1250 cP, no more than about 1500 cP, no more than about 1750 cP, no more than about 2000 cP, no more than about 2250 cP, or no more than about 2500 cP.

In some embodiments, the nasal spray has a viscosity of about 100 cP to about 2,500 cP. In some embodiments, the nasal spray has a viscosity of at least about 100 cP. In some embodiments, the nasal spray has a viscosity of at most about 2,500 cP. In some embodiments, the nasal spray has a viscosity of about 100 cP to about 250 cP, about 100 cP to about 500 cP, about 100 cP to about 750 cP, about 100 cP to about 1,000 cP, about 100 cP to about 1,250 cP, about 100 cP to about 1,500 cP, about 100 cP to about 1,750 cP, about 100 cP to about 2,000 cP, about 100 cP to about 2,250 cP, about 100 cP to about 2,500 cP, about 250 cP to about 500 cP, about 250 cP to about 750 cP, about 250 cP to about 1,000 cP, about 250 cP to about 1,250 cP, about 250 cP to about 1,500 cP, about 250 cP to about 1,750 cP, about 250 cP to about 2,000 cP, about 250 cP to about 2,250 cP, about 250 cP to about 2,500 cP, about 500 cP to about 750 cP, about 500 cP to about 1,000 cP, about 500 cP to about 1,250 cP, about 500 cP to about 1,500 cP, about 500 cP to about 1,750 cP, about 500 cP to about 2,000 cP, about 500 cP to about 2,250 cP, about 500 cP to about 2,500 cP, about 750 cP to about 1,000 cP, about 750 cP to about 1,250 cP, about 750 cP to about 1,500 cP, about 750 cP to about 1,750 cP, about 750 cP to about 2,000 cP, about 750 cP to about 2,250 cP, about 750 cP to about 2,500 cP, about 1,000 cP to about 1,250 cP, about 1,000 cP to about 1,500 cP, about 1,000 cP to about 1,750 cP, about 1,000 cP to about 2,000 cP, about 1,000 cP to about 2,250 cP, about 1,000 cP to about 2,500 cP, about 1,250 cP to about 1,500 cP, about 1,250 cP to about 1,750 cP, about 1,250 cP to about 2,000 cP, about 1,250 cP to about 2,250 cP, about 1,250 cP to about 2,500 cP, about 1,500 cP to about 1,750 cP, about 1,500 cP to about 2,000 cP, about 1,500 cP to about 2,250 cP, about 1,500 cP to about 2,500 cP, about 1,750 cP to about 2,000 cP, about 1,750 cP to about 2,250 cP, about 1,750 cP to about 2,500 cP, about 2,000 cP to about 2,250 cP, about 2,000 cP to about 2,500 cP, or about 2,250 cP to about 2,500 cP. In some embodiments, the nasal spray has a viscosity of about 100 cP, about 250 cP, about 500 cP, about 750 cP, about 1,000 cP, about 1,250 cP, about 1,500 cP, about 1,750 cP, about 2,000 cP, about 2,250 cP, or about 2,500 cP.

In some embodiments, the pharmaceutical composition comprises a tablet, capsule, or oral suspension. For example, the pharmaceutical composition comprises a tablet comprising from about 10 mg to about 1000 mg of Compound 1 or a pharmaceutically acceptable salt thereof.

### III. METHODS OF TREATING CANCER

### A. Method of Treating Cancer

The present invention provides a method of treating cancer in a subject in need thereof, comprising administering to the subject a therapeutically effective amount of a compound of Compound 1

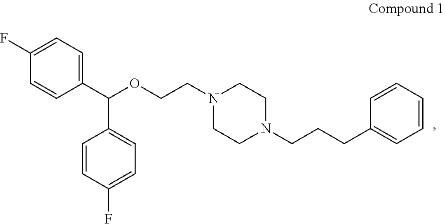 [SMILES: Fc1ccc(C(OCCN2CCN(CCCc3ccccc3)CC2)c2ccc(F)cc2)cc1]

or a pharmaceutically acceptable salt thereof, wherein the cancer is selected from brain cancer, breast cancer, pancreatic cancer, lung cancer, or any combination thereof.

In some implementations, Compound 1, or a pharmaceutically acceptable salt thereof, is a hydrochloride salt of Compound 1. For example, the hydrochloride salt is a dihydrochloride salt of Compound 1.

In some implementations, the cancer is brain cancer. Examples of brain cancer include, but are not limited to, acoustic neuroma, astrocytoma (e.g., piloid astrocytoma, fibrillary astrocytoma, protoplasmic astrocytoma, gemistocytic astrocytoma, anaplastic astrocytoma, and glioblastoma multiforme (GBM)), brain lymphoma, brain metastases, hypophyseal tumor (e.g., prolactinoma, HGH (human growth hormone) producing tumor, and ACTH-producing tumor (adrenocorticotrophic hormone)), craniopharyngiomas, medulloblastomas, meningiomas, and oligodendrogliomas. In some examples, the brain cancer is glioblastoma multiforme (GBM).

In some implementations, the cancer is breast cancer. Examples of breast cancer include, but are not limited to, mammary carcinoma, ductal carcinoma, colloid carcinoma, lobular invasive carcinoma, tubular carcinoma, adenoid cystic carcinoma, and papillary carcinoma.

In some implementations, the cancer is pancreatic cancer. Examples of pancreatic cancer include, but are not limited to, exocrine (nonendocrine) pancreatic cancer and neuroendocrine pancreatic cancer.

In some implementations, the cancer is lung cancer. Examples of lung cancer include, but are not limited to, bronchial carcinoma, small cell lung cancer, and non-small cell lung cancer (e.g., squamous epithelium carcinoma, lung adenocarcinoma, and large-cell bronchial carcinoma).

In some implementations, Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in cancer cells. In some implementations, Compound 1, or a pharmaceutically acceptable salt thereof, modulates activity of BMPR1A, UTP23, ARHGAP33, ITSN1, or any combination thereof in cancer cells. For example, Compound 1, or a pharmaceutically acceptable salt thereof, modulates activity of BMPR1A in cancer cells. In some examples, Compound 1, or a pharmaceutically acceptable salt thereof, modulates activity of UTP23 in cancer cells. In some examples, Compound 1, or a pharmaceutically acceptable salt thereof, modulates activity of ARHGAP33 in cancer cells. In some examples, Compound 1, or a pharmaceutically acceptable salt thereof, modulates activity of ITSN1 in cancer cells.

Compound 1, or a pharmaceutically acceptable salt thereof, that modulates BMPR1A, UTP23, ARHGAP33, ITSN1, CARD1, ETV2L, HMGN5, LYSMD4, PLA2G7, TAF11, TDRD5, UTP23, UGP2, WT1P, and/or ZNF75A function may be, for example, an agonist, partial agonist, antagonist, or partial antagonist of BMPR1A, UTP23, ARHGAP33, ITSN1, CARD1, ETV2L, HMGN5, LYSMD4, PLA2G7, TAF11, TDRD5, UTP23, UGP2, WT1P, and/or ZNF75A. Without wishing to be bound by theory, it is believed that Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in cancer cells and thereby exhibits cytotoxicity towards cancer cells. Compound 1 (also known as vanoxerine or GBR-12909) is a known dopamine reuptake inhibitor (DPI) that binds the target site of dopamine transporter (DAT). Izenwasser, S. et al., Comparison of the effects of cocaine and other inhibitors of dopamine uptake in rat striatum, nucleus accumbens, olfactory tubercle, and medial prefrontal cortex, 520 Brain Research 303 (1990). As such, it is believed that Compound 1 unexpectedly exhibits cytotoxicity towards cancer cells (e.g., GBM) cells via a novel mechanism of action.

In some implementations, Compound 1, or a pharmaceutically acceptable salt thereof, is administered as a pharmaceutical composition comprising a pharmaceutically acceptable carrier, excipient, or diluent. The pharmaceutically acceptable carrier, excipient, or diluent may be any carrier, excipient, or diluent described herein.

In some implementations, Compound 1, or a pharmaceutically acceptable salt thereof, is administered at least once per day. For example, Compound 1, or a pharmaceutically acceptable salt thereof, is administered once or twice per day. In some examples, Compound 1, or a pharmaceutically acceptable salt thereof, is administered once per day. In some examples, Compound 1, or a pharmaceutically acceptable salt thereof, is administered at least twice per day. For instance, Compound 1, or a pharmaceutically acceptable salt thereof, is administered twice or three times per day.

In some implementations, the method further comprises administering to the subject an additional therapeutic agent. For example, the additional therapeutic agent may be an anti-cancer agent. Examples of anti-cancer agents include, but are not limited to, abiraterone Acetate, afatinib, aldesleukin, alemtuzumab, alitretinoin, altretamine, amifostine, aminoglutethimide anagrelide, anastrozole, arsenic trioxide, asparaginase, azacitidine, azathioprine, bendamustine, bevacizumab, bexarotine, bicalutamide, bleomycin, bortezomib, busulfan, capecitabine, carboplatin, carmustine, cemiplimab, cetuximab, chlorambucil, cisplatin, cladribine, crizotinib, cyclophosphamide, cytarabine, dacarbazine, dactinomycin, dasatinib, daunorubicin, denileukin diftitox, decitabine, docetaxel, dexamethasone, dostarlimab, doxifluridine, doxorubicin, enzalutamide, epirubicin, epoetin alpha, epothilones, erlotinib, erubulin, estramustine, etinostat, etoposide, everolimus, exemestane, filgrastim, floxuridine, fludarabine, fluorouracil, fluoxymesterone, flutamide, folate linked alkaloids, gefitinib, gemcitabine, gemtuzumab ozogamicin, GM-CT-01, goserelin, hexamethylmelamine, hydroxyureas, ibritumomab, idarubicin, ifosfamide, imatinib, interferon alpha, interferon beta, irinotecan, ixabepilone, lapatinib, latrunculin, leucovorin, leuprolide, lenalidomide, letrozole, lomustine, mechlorethamine, megestrol, melphalan, mercaptopurine, methotrexate, mitomycin, mitoxantrone, nelarabine, nilotinib, nilutamide, nivolumab, octreotide, ofatumumab, omacetaxine mepesuccinate, oprelvekin, oxaliplatin, paclitaxel, panitumumab, pembrolizumab, pemetrexed, pentostatin, polysaccharide galectin inhibitors, procarbazine, proscillaridin A, raloxifene, retinoic acids, rituximab, romiplostim, sargramostim, sorafenib, streptozocin, sunitinib, tamoxifen, temsirolimus, temozolomide, teniposide, thalidomide, thioguanine, thiotepa, tioguanine, topotecan, toremifene, tositumomab, trametinib, trastuzumab, tretinoin, valrubicin, VEGF inhibitors and traps, vinblastine, vincristine, vindesine, vinorelbine, vintafolide (EC145), and vorinostat.

In some implementations, the anti-cancer agent is selected from temozolomide, ixabepilone, cladribine, enzalutamide, omacetaxine mepesuccinate, epothilones, erubulin, latrunculin, a pharmaceutically acceptable salt of any of these anti-cancer agents, or any combination thereof. For example, the anti-cancer agent is temozolomide, or a pharmaceutically acceptable salt thereof. In some examples, the anti-cancer agent is ixabepilone, or a pharmaceutically acceptable salt thereof. In some examples, the anti-cancer agent is cladribine, or a pharmaceutically acceptable salt thereof. In some examples, the anti-cancer agent is enzalutamide, or a pharmaceutically acceptable salt thereof. In some examples, the anti-cancer agent is omacetaxine mepesuccinate, or a pharmaceutically acceptable salt thereof. In some examples, the anti-cancer agent is epothilones, or a pharmaceutically acceptable salt thereof. In some examples, the anti-cancer agent is erubulin, or a pharmaceutically acceptable salt thereof. And, in some examples, the anti-cancer agent is latrunculin, or a pharmaceutically acceptable salt thereof.

In some implementations, Compound 1, or a pharmaceutically acceptable salt thereof, and the additional therapeutic agent are administered concurrently. In other implementations, Compound 1, or a pharmaceutically acceptable salt thereof, and the additional therapeutic agent are administered sequentially. For example, the additional therapeutic agent may be administered prior to or subsequent to Compound 1, or a pharmaceutically acceptable salt thereof.

In some implementations, the method further comprises administering to the subject a radiation therapy. In other words, radiation therapy may be administered during the treatment course wherein a compound of the present invention (or a pharmaceutically acceptable salt thereof) is administered to a patient in need thereof.

In some implementations, Compound 1, or a pharmaceutically acceptable salt thereof, is administered in a dosage amount of from about 1 mg to about 2,000 mg. For example, Compound 1, or a pharmaceutically acceptable salt thereof, is administered in a dosage amount of from about 1 mg to about 1,000 mg. In some examples, Compound 1, or a pharmaceutically acceptable salt thereof, is administered in a dosage amount of from about 1 mg to about 900 mg, 1 mg to about 800 mg, 1 mg to about 700 mg, 1 mg to about 600 mg, 1 mg to about 500 mg, 1 mg to about 400 mg, 1 mg to about 300 mg, 1 mg to about 200 mg, 1 mg to about 100 mg, 1 mg to about 75 mg, or 1 mg to about 50 mg. In some examples, Compound 1, or a pharmaceutically acceptable salt thereof, is administered in a dosage amount of from about 50 mg to about 100 mg. In some examples, Compound 1, or a pharmaceutically acceptable salt thereof, is administered in a dosage amount of from about 100 mg to about 500 mg. In some examples, Compound 1, or a pharmaceutically acceptable salt thereof, is administered in a dosage amount of from about 50 mg to about 500 mg. In some examples, Compound 1, or a pharmaceutically acceptable salt thereof, is administered in a dosage amount of from about 50 mg to about 300 mg. In some examples, Compound 1, or a pharmaceutically acceptable salt thereof, is administered in a dosage amount of from about 75 mg to about 300 mg. In some examples, Compound 1, or a pharmaceutically acceptable salt thereof, is administered in a dosage amount of from about 100 mg to about 300 mg. In some examples, Compound 1, or a pharmaceutically acceptable salt thereof, is administered in a dosage amount of from about 75 mg to about 150 mg. In some examples, Compound 1, or a pharmaceutically acceptable salt thereof, is administered in a dosage amount of about 50 mg. In some examples, Compound 1, or a pharmaceutically acceptable salt thereof, is administered in a dosage amount of about 75 mg. In some examples, Compound 1, or a pharmaceutically acceptable salt thereof, is administered in a dosage amount of about 100 mg. In some examples, Compound 1, or a pharmaceutically acceptable salt thereof, is administered in a dosage amount of about 200 mg. In some examples, Compound 1, or a pharmaceutically acceptable salt thereof, is administered in a dosage amount of about 300 mg. And, in some examples, Compound 1, or a pharmaceutically acceptable salt thereof, is administered in a dosage amount of about 400 mg.

In some implementations, Compound 1, or a pharmaceutically acceptable salt thereof, is administered chronically (i.e., “chronic administration”). Chronic administration refers to administration Compound 1 or a pharmaceutically acceptable salt thereof, or a pharmaceutical composition thereof, over an extended period of time (e.g., over 3 months, 6 months, 1 year, 2 years, 3 years, 5 years, etc.), or indefinitely (e.g., for the rest of the subject's life). In some examples, the chronic administration is intended to provide a constant level of Compound 1 or a pharmaceutically acceptable salt thereof in the blood (e.g., within the therapeutic window over the extended period of time).

In some implementations, Compound 1, or a pharmaceutically acceptable salt thereof, is administered at least once a day from onset of treatment for a duration of at least about 2 days. For example, Compound 1, or a pharmaceutically acceptable salt thereof, is administered at least once a day from onset of treatment for a duration of at least about 5 days, 10 days, 15 days, 20 days, 25 days, 30 days, 60 days, 90 days, 120 days, 180 days, or 1 year. In some examples, Compound 1, or a pharmaceutically acceptable salt thereof, is administered at least once a day from onset of treatment for a duration of at least about 1 month, 2 months, 3 months, 4 months, 5 months, 6 months, 7, months, 8 months, 9 months, 10 months, or 11 months. In some examples, Compound 1, or a pharmaceutically acceptable salt thereof, is administered at least once a day from onset of treatment for a duration of at least about 1 year. And, in some examples, Compound 1, or a pharmaceutically acceptable salt thereof, is administered at least once a day from onset of treatment for a duration of at least about 2 years, 3 years, 4 years, 5 years, 6 years, 7 years, 8 years, 9 years, or 10 years.

In some implementations, Compound 1, or a pharmaceutically acceptable salt thereof, is administered orally, subcutaneously, intramuscularly, intravenously, intracranially, intrathecally, or intranasally. For example, Compound 1, or a pharmaceutically acceptable salt thereof, is administered orally or intranasally. In some examples, Compound 1, or a pharmaceutically acceptable salt thereof, is administered orally. For example, Compound 1, or a pharmaceutically acceptable salt thereof, is administered as a tablet, capsule, or oral suspension. In some examples, Compound 1, or a pharmaceutically acceptable salt thereof, is administered intranasally. For example, Compound 1, or a pharmaceutically acceptable salt thereof, is administered as a nasal spray.

In some implementations, the subject experiences a reduced loss in bodyweight following treatment onset as compared to a subject not administered Compound 1, or a pharmaceutically acceptable salt thereof. For example, the subject experiences a reduced loss in bodyweight of at least about 1% (e.g., at least about 2%, at least about 3%, at least about 4%, at least about 5%, at least about 6%, at least about 7%, at least about 8%, or at least about 9%) following treatment onset as compared to a subject not administered Compound 1, or a pharmaceutically acceptable salt thereof. In other examples, the subject experiences a reduced loss in bodyweight of at least about 10% (e.g., at least about 11%, at least about 12%, at least about 13%, at least about 14%, at least about 15%, at least about 16%, at least about 17%, at least about 18%, or at least about 19%) following treatment onset as compared to a subject not administered Compound 1, or a pharmaceutically acceptable salt thereof. In some examples, the subject experiences a reduced loss in bodyweight of at least about 20% (e.g., at least about 21%, at least about 22%, at least about 23%, at least about 24%, at least about 25%, at least about 26%, at least about 27%, at least about 28%, or at least about 29%) following treatment onset as compared to a subject not administered Compound 1, or a pharmaceutically acceptable salt thereof. And, in some examples, the subject experiences a reduced loss in bodyweight of at least about 30% to about 99% (e.g., from about 30% to about 85%, from about 30% to about 80%, from about 30% to about 75%, and the like) following treatment onset as compared to a subject not administered Compound 1, or a pharmaceutically acceptable salt thereof.

In some implementations, the subject experiences an increased life expectancy following treatment onset as compared to a subject not administered Compound 1, or a pharmaceutically acceptable salt thereof. For example, the subject experiences an increased life expectancy of at least about 1% (e.g., at least about 2%, at least about 3%, at least about 4%, at least about 5%, at least about 6%, at least about 7%, at least about 8%, or at least about 9%) following treatment onset as compared to a subject not administered Compound 1, or a pharmaceutically acceptable salt thereof. In other examples, the subject experiences an increased life expectancy of at least about 10% (e.g., at least about 11%, at least about 12%, at least about 13%, at least about 14%, at least about 15%, at least about 16%, at least about 17%, at least about 18%, or at least about 19%) following treatment onset as compared to a subject not administered Compound 1, or a pharmaceutically acceptable salt thereof. In some examples, the subject experiences an increased life expectancy of at least about 20% (e.g., at least about 21%, at least about 22%, at least about 23%, at least about 24%, at least about 25%, at least about 26%, at least about 27%, at least about 28%, or at least about 29%) following treatment onset as compared to a subject not administered Compound 1, or a pharmaceutically acceptable salt thereof. And, in some examples, the subject experiences an increased life expectancy of at least about 30% to about 99% (e.g., from about 30% to about 85%, from about 30% to about 80%, from about 30% to about 75%, and the like) following treatment onset as compared to a subject not administered Compound 1, or a pharmaceutically acceptable salt thereof.

In some implementations, the subject experiences a vacuolization of mitochondria in cancer cells following treatment onset. In other implementations, the subject experiences a depolarization of mitochondria in cancer cells following treatment onset.

Without wishing to be bound by theory, it is believed that treatment with Compound 1, or a pharmaceutically acceptable salt thereof, induces vacuolization of mitochondria in cancer cells (e.g., glioblastoma multiforme (GBM) cells). This vacuolization results in depolarization of mitochondria in cancer cells and, ultimately, cell death.

In some implementations, the subject is an animal. For example, the subject is a mammal. In other examples, the subject is a human.

### B. Method of Treating Glioma

Another aspect of the present invention provides a method of treating glioma in a subject in need thereof, comprising administering to the subject a therapeutically effective amount of Compound 1

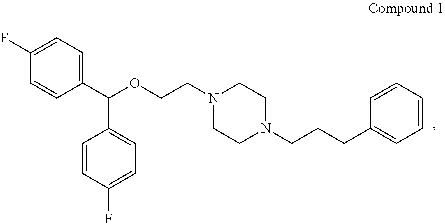 [SMILES: Fc1ccc(C(OCCN2CCN(CCCc3ccccc3)CC2)c2ccc(F)cc2)cc1]

or a pharmaceutically acceptable salt thereof.

In some implementations, Compound 1, or a pharmaceutically acceptable salt thereof, is a hydrochloride salt. For example, the hydrochloride salt is a dihydrochloride salt.

In some implementations, the glioma is selected from the group consisting of an astrocytoma, an ependymoma, an oligodendroglioma, a brainstem glioma, an optic nerve glioma, and a mixed glioma. For example, the glioma is an astrocytoma. Astroctyoma includes, by way of non-limiting example, pilocytic astrocytoma, subependymal giant cell astrocytoma, fibrillary astrocytoma, pleomorphic xanthoastrocytoma, mixed oligoastrocytoma, anaplastic astrocytoma, and glioblastoma multiforme (GBM). In some examples, the astrocytoma is GBM.

In some examples, the glioma is ependymoma. Ependymoma includes, by way of non-limiting example, subependymoma, myxopapillary ependymoma, and anaplastic ependymoma.

In some examples, the glioma is an oligodendroglioma. Oligodendroglioma includes, by way of non-limiting example, Grade II oligodendroglioma and Grade III oligodendroglioma (i.e., anaplastic oligodendrioglioma).

In some examples, the glioma is a brainstem glioma. Brainstem glioma includes, by way of non-limiting example, tectal Glioma, diffuse intrinsic pontine glioma (DIPG), and cervicomedullary glioma.

In some examples, the glioma is an optic nerve glioma. In some examples, the glioma is a mixed glioma (e.g., oligodendroglioma & oligoastrocytoma).

In some implementations, Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in glioma cells (e.g., GBM cells). In some implementations, Compound 1, or a pharmaceutically acceptable salt thereof, modulates activity of BMPR1A, UTP23, ARHGAP33, ITSN1, or any combination thereof in glioma cells (e.g., GBM cells). For example, Compound 1, or a pharmaceutically acceptable salt thereof, modulates activity of BMPR1A in glioma cells. In some examples, Compound 1, or a pharmaceutically acceptable salt thereof, modulates activity of UTP23 in glioma cells. In some examples, Compound 1, or a pharmaceutically acceptable salt thereof, modulates activity of ARHGAP33 in glioma cells. In some examples, Compound 1, or a pharmaceutically acceptable salt thereof, modulates activity of ITSN1 in glioma cells.

In some implementations, Compound 1, or a pharmaceutically acceptable salt thereof, is administered as a pharmaceutical composition comprising a pharmaceutically acceptable carrier, excipient, or diluent. The pharmaceutically acceptable carrier, excipient, or diluent may be any carrier, excipient, or diluent described herein.

In some implementations, Compound 1, or a pharmaceutically acceptable salt thereof, is administered at least once per day. For example, Compound 1, or a pharmaceutically acceptable salt thereof, is administered once or twice per day. In some examples, Compound 1, or a pharmaceutically acceptable salt thereof, is administered once per day.

In some implementations, the method further comprises administering to the subject an additional therapeutic agent. For example, the additional therapeutic agent may be an anti-cancer agent. Examples of anti-cancer agents include, but are not limited to, abiraterone Acetate, afatinib, aldesleukin, alemtuzumab, alitretinoin, altretamine, amifostine, aminoglutethimide anagrelide, anastrozole, arsenic trioxide, asparaginase, azacitidine, azathioprine, bendamustine, bevacizumab, bexarotine, bicalutamide, bleomycin, bortezomib, busulfan, capecitabine, carboplatin, carmustine, cemiplimab, cetuximab, chlorambucil, cisplatin, cladribine, crizotinib, cyclophosphamide, cytarabine, dacarbazine, dactinomycin, dasatinib, daunorubicin, denileukin diftitox, decitabine, docetaxel, dexamethasone, dostarlimab, doxifluridine, doxorubicin, enzalutamide, epirubicin, epoetin alpha, epothilones, erlotinib, erubulin, estramustine, etinostat, etoposide, everolimus, exemestane, filgrastim, floxuridine, fludarabine, fluorouracil, fluoxymesterone, flutamide, folate linked alkaloids, gefitinib, gemcitabine, gemtuzumab ozogamicin, GM-CT-01, goserelin, hexamethylmelamine, hydroxyureas, ibritumomab, idarubicin, ifosfamide, imatinib, interferon alpha, interferon beta, irinotecan, ixabepilone, lapatinib, latrunculin, leucovorin, leuprolide, lenalidomide, letrozole, lomustine, mechlorethamine, megestrol, melphalan, mercaptopurine, methotrexate, mitomycin, mitoxantrone, nelarabine, nilotinib, nilutamide, nivolumab, octreotide, ofatumumab, omacetaxine mepesuccinate, oprelvekin, oxaliplatin, paclitaxel, panitumumab, pembrolizumab, pemetrexed, pentostatin, polysaccharide galectin inhibitors, procarbazine, proscillaridin A, raloxifene, retinoic acids, rituximab, romiplostim, sargramostim, sorafenib, streptozocin, sunitinib, tamoxifen, temsirolimus, temozolomide, teniposide, thalidomide, thioguanine, thiotepa, tioguanine, topotecan, toremifene, tositumomab, trametinib, trastuzumab, tretinoin, valrubicin, VEGF inhibitors and traps, vinblastine, vincristine, vindesine, vinorelbine, vintafolide (EC145), and vorinostat.

In some implementations, the anti-cancer agent is selected from temozolomide, ixabepilone, cladribine, enzalutamide, omacetaxine mepesuccinate, epothilones, erubulin, latrunculin, a pharmaceutically acceptable salt of any of these anti-cancer agents, or any combination thereof. For example, the anti-cancer agent is temozolomide, or a pharmaceutically acceptable salt thereof. In some examples, the anti-cancer agent is ixabepilone, or a pharmaceutically acceptable salt thereof. In some examples, the anti-cancer agent is cladribine, or a pharmaceutically acceptable salt thereof. In some examples, the anti-cancer agent is enzalutamide, or a pharmaceutically acceptable salt thereof. In some examples, the anti-cancer agent is omacetaxine mepesuccinate, or a pharmaceutically acceptable salt thereof. In some examples, the anti-cancer agent is epothilones, or a pharmaceutically acceptable salt thereof. In some examples, the anti-cancer agent is erubulin, or a pharmaceutically acceptable salt thereof. And, in some examples, the anti-cancer agent is latrunculin, or a pharmaceutically acceptable salt thereof.

In some implementations, Compound 1, or a pharmaceutically acceptable salt thereof, and the additional therapeutic agent are administered concurrently. In other implementations, Compound 1, or a pharmaceutically acceptable salt thereof, and the additional therapeutic agent are administered sequentially. For example, the additional therapeutic agent may be administered prior to, or subsequent to, Compound 1, or a pharmaceutically acceptable salt thereof.

In some implementations, the method further comprises administering to the subject a radiation therapy. In other words, radiation therapy may be administered during the treatment course wherein a compound of the present invention (or a pharmaceutically acceptable salt thereof) is administered to a patient in need thereof.

In some implementations, Compound 1, or a pharmaceutically acceptable salt thereof, is administered in a dosage amount of from about 1 mg to about 2,000 mg. For example, Compound 1, or a pharmaceutically acceptable salt thereof, is administered in a dosage amount of from about 1 mg to about 1,000 mg. In some examples, Compound 1, or a pharmaceutically acceptable salt thereof, is administered in a dosage amount of from about 1 mg to about 900 mg, 1 mg to about 800 mg, 1 mg to about 700 mg, 1 mg to about 600 mg, 1 mg to about 500 mg, 1 mg to about 400 mg, 1 mg to about 300 mg, 1 mg to about 200 mg, 1 mg to about 100 mg, 1 mg to about 75 mg, or 1 mg to about 50 mg. In some examples, Compound 1, or a pharmaceutically acceptable salt thereof, is administered in a dosage amount of from about 50 mg to about 100 mg. In some examples, Compound 1, or a pharmaceutically acceptable salt thereof, is administered in a dosage amount of from about 100 mg to about 500 mg. In some examples, Compound 1, or a pharmaceutically acceptable salt thereof, is administered in a dosage amount of from about 50 mg to about 500 mg. In some examples, Compound 1, or a pharmaceutically acceptable salt thereof, is administered in a dosage amount of from about 50 mg to about 300 mg. In some examples, Compound 1, or a pharmaceutically acceptable salt thereof, is administered in a dosage amount of from about 75 mg to about 300 mg. In some examples, Compound 1, or a pharmaceutically acceptable salt thereof, is administered in a dosage amount of from about 100 mg to about 300 mg. In some examples, Compound 1, or a pharmaceutically acceptable salt thereof, is administered in a dosage amount of from about 75 mg to about 150 mg. In some examples, Compound 1, or a pharmaceutically acceptable salt thereof, is administered in a dosage amount of about 50 mg. In some examples, Compound 1, or a pharmaceutically acceptable salt thereof, is administered in a dosage amount of about 75 mg. In some examples, Compound 1, or a pharmaceutically acceptable salt thereof, is administered in a dosage amount of about 100 mg. In some examples, Compound 1, or a pharmaceutically acceptable salt thereof, is administered in a dosage amount of about 200 mg. In some examples, Compound 1, or a pharmaceutically acceptable salt thereof, is administered in a dosage amount of about 300 mg. And, in some examples, Compound 1, or a pharmaceutically acceptable salt thereof, is administered in a dosage amount of about 400 mg.

In some implementations, Compound 1, or a pharmaceutically acceptable salt thereof, is administered chronically.

In some implementations, Compound 1, or a pharmaceutically acceptable salt thereof, is administered at least once a day from onset of treatment for a duration of at least about 2 days. For example, Compound 1, or a pharmaceutically acceptable salt thereof, is administered at least once a day from onset of treatment for a duration of at least about 5 days, 10 days, 15 days, 20 days, 25 days, 30 days, or 60 days. In some examples, Compound 1, or a pharmaceutically acceptable salt thereof, is administered at least once a day from onset of treatment for a duration of at least about 1 month, 2 months, 3 months, 4 months, 5 months, 6 months, 7, months, 8 months, 9 months, 10 months, or 11 months. In some examples, Compound 1, or a pharmaceutically acceptable salt thereof, is administered at least once a day from onset of treatment for a duration of at least about 1 year. And, in some examples, Compound 1, or a pharmaceutically acceptable salt thereof, is administered at least once a day from onset of treatment for a duration of at least about 2 years, 3 years, 4 years, 5 years, 6 years, 7 years, 8 years, 9 years, or 10 years.

In some implementations, Compound 1, or a pharmaceutically acceptable salt thereof, is administered orally, subcutaneously, intramuscularly, intravenously, intracranially, intrathecally, or intranasally. For example, Compound 1, or a pharmaceutically acceptable salt thereof, is administered orally or intranasally. In some examples, Compound 1, or a pharmaceutically acceptable salt thereof, is administered orally. For example, Compound 1, or a pharmaceutically acceptable salt thereof, is administered as a tablet, capsule, or oral suspension. In some examples, Compound 1, or a pharmaceutically acceptable salt thereof, is administered intranasally. For example, Compound 1, or a pharmaceutically acceptable salt thereof, is administered as a nasal spray.

In some implementations, the subject experiences a reduced loss in bodyweight following treatment onset as compared to a subject not administered Compound 1, or a pharmaceutically acceptable salt thereof. For example, the subject experiences a reduced loss in bodyweight of at least about 1% (e.g., at least about 2%, at least about 3%, at least about 4%, at least about 5%, at least about 6%, at least about 7%, at least about 8%, or at least about 9%) following treatment onset as compared to a subject not administered Compound 1, or a pharmaceutically acceptable salt thereof. In other examples, the subject experiences a reduced loss in bodyweight of at least about 10% (e.g., at least about 11%, at least about 12%, at least about 13%, at least about 14%, at least about 15%, at least about 16%, at least about 17%, at least about 18%, or at least about 19%) following treatment onset as compared to a subject not administered Compound 1, or a pharmaceutically acceptable salt thereof. In some examples, the subject experiences a reduced loss in bodyweight of at least about 20% (e.g., at least about 21%, at least about 22%, at least about 23%, at least about 24%, at least about 25%, at least about 26%, at least about 27%, at least about 28%, or at least about 29%) following treatment onset as compared to a subject not administered Compound 1, or a pharmaceutically acceptable salt thereof. And, in some examples, the subject experiences a reduced loss in bodyweight of at least about 30% to about 99% (e.g., from about 30% to about 85%, from about 30% to about 80%, from about 30% to about 75%, and the like) following treatment onset as compared to a subject not administered Compound 1, or a pharmaceutically acceptable salt thereof.

In some implementations, the subject experiences an increased life expectancy following treatment onset as compared to a subject not administered Compound 1, or a pharmaceutically acceptable salt thereof. For example, the subject experiences an increased life expectancy of at least about 1% (e.g., at least about 2%, at least about 3%, at least about 4%, at least about 5%, at least about 6%, at least about 7%, at least about 8%, or at least about 9%) following treatment onset as compared to a subject not administered Compound 1, or a pharmaceutically acceptable salt thereof. In other examples, the subject experiences an increased life expectancy of at least about 10% (e.g., at least about 11%, at least about 12%, at least about 13%, at least about 14%, at least about 15%, at least about 16%, at least about 17%, at least about 18%, or at least about 19%) following treatment onset as compared to a subject not administered Compound 1, or a pharmaceutically acceptable salt thereof. In some examples, the subject experiences an increased life expectancy of at least about 20% (e.g., at least about 21%, at least about 22%, at least about 23%, at least about 24%, at least about 25%, at least about 26%, at least about 27%, at least about 28%, or at least about 29%) following treatment onset as compared to a subject not administered Compound 1, or a pharmaceutically acceptable salt thereof. And, in some examples, the subject experiences an increased life expectancy of at least about 30% to about 99% (e.g., from about 30% to about 85%, from about 30% to about 80%, from about 30% to about 75%, and the like) following treatment onset as compared to a subject not administered Compound 1, or a pharmaceutically acceptable salt thereof.

In some implementations, the subject experiences a vacuolization of mitochondria in glioma cells (e.g., GBM cells) following treatment onset. In other implementations, the subject experiences a depolarization of mitochondria in glioma cells (e.g., GBM cells) following treatment onset.

In some implementations, the subject is an animal. For example, the subject is a human.

### C. Method of Inducing Vacuolization

Another aspect of the present invention provides a method of inducing vacuolization of mitochondria in glioblastoma multiforme (GBM) cells of a subject, comprising administering to the subject a therapeutically effective amount of Compound 1

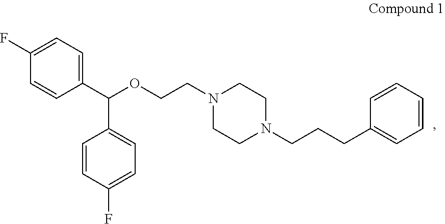 [SMILES: Fc1ccc(C(OCCN2CCN(CCCc3ccccc3)CC2)c2ccc(F)cc2)cc1]

or a pharmaceutically acceptable salt thereof.

In some implementations, Compound 1, or a pharmaceutically acceptable salt thereof, is a hydrochloride salt. For example, the hydrochloride salt is a dihydrochloride salt.

In some implementations, Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in GBM cells. In some implementations, Compound 1, or a pharmaceutically acceptable salt thereof, modulates activity of BMPR1A, UTP23, ARHGAP33, ITSN1, or any combination thereof in GBM cells. For example, Compound 1, or a pharmaceutically acceptable salt thereof, modulates activity of BMPR1A in GBM cells. In some examples, Compound 1, or a pharmaceutically acceptable salt thereof, modulates activity of UTP23 in GBM cells. In some examples, Compound 1, or a pharmaceutically acceptable salt thereof, modulates activity of ARHGAP33 in GBM cells. In some examples, Compound 1, or a pharmaceutically acceptable salt thereof, modulates activity of ITSN1 in GBM cells.

In some implementations, Compound 1, or a pharmaceutically acceptable salt thereof, is administered as a pharmaceutical composition comprising a pharmaceutically acceptable carrier, excipient, or diluent. The pharmaceutically acceptable carrier, excipient, or diluent may be any carrier, excipient, or diluent described herein.

In some implementations, Compound 1, or a pharmaceutically acceptable salt thereof, is administered at least once per day. For example, Compound 1, or a pharmaceutically acceptable salt thereof, is administered once or twice per day. In some examples, Compound 1, or a pharmaceutically acceptable salt thereof, is administered once per day.

In some implementations, Compound 1, or a pharmaceutically acceptable salt thereof, is administered in a dosage amount of from about 1 mg to about 2,000 mg. For example, Compound 1, or a pharmaceutically acceptable salt thereof, is administered in a dosage amount of from about 1 mg to about 1,000 mg. In some examples, Compound 1, or a pharmaceutically acceptable salt thereof, is administered in a dosage amount of from about 1 mg to about 900 mg, 1 mg to about 800 mg, 1 mg to about 700 mg, 1 mg to about 600 mg, 1 mg to about 500 mg, 1 mg to about 400 mg, 1 mg to about 300 mg, 1 mg to about 200 mg, 1 mg to about 100 mg, 1 mg to about 75 mg, or 1 mg to about 50 mg. In some examples, Compound 1, or a pharmaceutically acceptable salt thereof, is administered in a dosage amount of from about 50 mg to about 100 mg. In some examples, Compound 1, or a pharmaceutically acceptable salt thereof, is administered in a dosage amount of from about 100 mg to about 500 mg. In some examples, Compound 1, or a pharmaceutically acceptable salt thereof, is administered in a dosage amount of from about 50 mg to about 500 mg. In some examples, Compound 1, or a pharmaceutically acceptable salt thereof, is administered in a dosage amount of from about 50 mg to about 300 mg. In some examples, Compound 1, or a pharmaceutically acceptable salt thereof, is administered in a dosage amount of from about 75 mg to about 300 mg. In some examples, Compound 1, or a pharmaceutically acceptable salt thereof, is administered in a dosage amount of from about 100 mg to about 300 mg. In some examples, Compound 1, or a pharmaceutically acceptable salt thereof, is administered in a dosage amount of from about 75 mg to about 150 mg. In some examples, Compound 1, or a pharmaceutically acceptable salt thereof, is administered in a dosage amount of about 50 mg. In some examples, Compound 1, or a pharmaceutically acceptable salt thereof, is administered in a dosage amount of about 75 mg. In some examples, Compound 1, or a pharmaceutically acceptable salt thereof, is administered in a dosage amount of about 100 mg. In some examples, Compound 1, or a pharmaceutically acceptable salt thereof, is administered in a dosage amount of about 200 mg. In some examples, Compound 1, or a pharmaceutically acceptable salt thereof, is administered in a dosage amount of about 300 mg. And, in some examples, Compound 1, or a pharmaceutically acceptable salt thereof, is administered in a dosage amount of about 400 mg.

In some implementations, Compound 1, or a pharmaceutically acceptable salt thereof, is administered chronically.

In some implementations, Compound 1, or a pharmaceutically acceptable salt thereof, is administered at least once a day from onset of treatment for a duration of at least about 2 days. For example, Compound 1, or a pharmaceutically acceptable salt thereof, is administered at least once a day from onset of treatment for a duration of at least about 5 days, 10 days, 15 days, 20 days, 25 days, 30 days, or 60 days. In some examples, Compound 1, or a pharmaceutically acceptable salt thereof, is administered at least once a day from onset of treatment for a duration of at least about 1 month, 2 months, 3 months, 4 months, 5 months, 6 months, 7, months, 8 months, 9 months, 10 months, or 11 months. In some examples, Compound 1, or a pharmaceutically acceptable salt thereof, is administered at least once a day from onset of treatment for a duration of at least about 1 year. And, in some examples, Compound 1, or a pharmaceutically acceptable salt thereof, is administered at least once a day from onset of treatment for a duration of at least about 2 years, 3 years, 4 years, 5 years, 6 years, 7 years, 8 years, 9 years, or 10 years.

In some implementations, Compound 1, or a pharmaceutically acceptable salt thereof, is administered orally, subcutaneously, intramuscularly, intravenously, intracranially, intrathecally, or intranasally. For example, Compound 1, or a pharmaceutically acceptable salt thereof, is administered orally or intranasally. In some examples, Compound 1, or a pharmaceutically acceptable salt thereof, is administered orally. For example, Compound 1, or a pharmaceutically acceptable salt thereof, is administered as a tablet, capsule, or oral suspension. In some examples, Compound 1, or a pharmaceutically acceptable salt thereof, is administered intranasally. For example, Compound 1, or a pharmaceutically acceptable salt thereof, is administered as a nasal spray.

In some implementations, the subject experiences a depolarization of mitochondria in GBM cells following treatment onset.

In some implementations, the subject is an animal. For example, the subject is a human.

### D. Method of Depolarizing Mitochondria

Another aspect of the present invention provides a method of depolarizing mitochondria in glioblastoma multiforme (GBM) cells of a subject, comprising administering to the subject a therapeutically effective amount of Compound 1

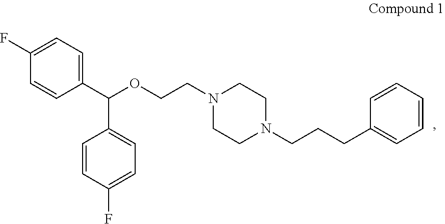 [SMILES: Fc1ccc(C(OCCN2CCN(CCCc3ccccc3)CC2)c2ccc(F)cc2)cc1]

or a pharmaceutically acceptable salt thereof.

In some implementations, Compound 1, or a pharmaceutically acceptable salt thereof, is a hydrochloride salt. For example, the hydrochloride salt is a dihydrochloride salt.

In some implementations, Compound 1, or a pharmaceutically acceptable salt thereof, modulates activity of BMPR1A, UTP23, ARHGAP33, ITSN1, CARD1, ETV2L, HMGN5, LYSMD4, PLA2G7, TAF11, TDRD5, UTP23, UGP2, WT1P, ZNF75A, or any combination thereof in GBM cells. In some implementations, Compound 1, or a pharmaceutically acceptable salt thereof, modulates activity of BMPR1A, UTP23, ARHGAP33, ITSN1, or any combination thereof in GBM cells. For example, Compound 1, or a pharmaceutically acceptable salt thereof, modulates activity of BMPR1A in GBM cells. In some examples, Compound 1, or a pharmaceutically acceptable salt thereof, modulates activity of UTP23 in GBM cells. In some examples, Compound 1, or a pharmaceutically acceptable salt thereof, modulates activity of ARHGAP33 in GBM cells. In some examples, Compound 1, or a pharmaceutically acceptable salt thereof, modulates activity of ITSN1 in GBM cells.

In some implementations, Compound 1, or a pharmaceutically acceptable salt thereof, is administered as a pharmaceutical composition comprising a pharmaceutically acceptable carrier, excipient, or diluent. The pharmaceutically acceptable carrier, excipient, or diluent may be any carrier, excipient, or diluent described herein.

In some implementations, Compound 1, or a pharmaceutically acceptable salt thereof, is administered at least once per day. For example, Compound 1, or a pharmaceutically acceptable salt thereof, is administered once or twice per day. In some examples, Compound 1, or a pharmaceutically acceptable salt thereof, is administered once per day.

In some implementations, Compound 1, or a pharmaceutically acceptable salt thereof, is administered in a dosage amount of from about 1 mg to about 2,000 mg. For example, Compound 1, or a pharmaceutically acceptable salt thereof, is administered in a dosage amount of from about 1 mg to about 1,000 mg. In some examples, Compound 1, or a pharmaceutically acceptable salt thereof, is administered in a dosage amount of from about 1 mg to about 900 mg, 1 mg to about 800 mg, 1 mg to about 700 mg, 1 mg to about 600 mg, 1 mg to about 500 mg, 1 mg to about 400 mg, 1 mg to about 300 mg, 1 mg to about 200 mg, 1 mg to about 100 mg, 1 mg to about 75 mg, or 1 mg to about 50 mg. In some examples, Compound 1, or a pharmaceutically acceptable salt thereof, is administered in a dosage amount of from about 50 mg to about 100 mg. In some examples, Compound 1, or a pharmaceutically acceptable salt thereof, is administered in a dosage amount of from about 100 mg to about 500 mg. In some examples, Compound 1, or a pharmaceutically acceptable salt thereof, is administered in a dosage amount of from about 50 mg to about 500 mg. In some examples, Compound 1, or a pharmaceutically acceptable salt thereof, is administered in a dosage amount of from about 50 mg to about 300 mg. In some examples, Compound 1, or a pharmaceutically acceptable salt thereof, is administered in a dosage amount of from about 75 mg to about 300 mg. In some examples, Compound 1, or a pharmaceutically acceptable salt thereof, is administered in a dosage amount of from about 100 mg to about 300 mg. In some examples, Compound 1, or a pharmaceutically acceptable salt thereof, is administered in a dosage amount of from about 75 mg to about 150 mg. In some examples, Compound 1, or a pharmaceutically acceptable salt thereof, is administered in a dosage amount of about 50 mg. In some examples, Compound 1, or a pharmaceutically acceptable salt thereof, is administered in a dosage amount of about 75 mg. In some examples, Compound 1, or a pharmaceutically acceptable salt thereof, is administered in a dosage amount of about 100 mg. In some examples, Compound 1, or a pharmaceutically acceptable salt thereof, is administered in a dosage amount of about 200 mg. In some examples, Compound 1, or a pharmaceutically acceptable salt thereof, is administered in a dosage amount of about 300 mg. And, in some examples, Compound 1, or a pharmaceutically acceptable salt thereof, is administered in a dosage amount of about 400 mg.

In some implementations, Compound 1, or a pharmaceutically acceptable salt thereof, is administered chronically.

In some implementations, Compound 1, or a pharmaceutically acceptable salt thereof, is administered at least once a day from onset of treatment for a duration of at least about 2 days. For example, Compound 1, or a pharmaceutically acceptable salt thereof, is administered at least once a day from onset of treatment for a duration of at least about 5 days, 10 days, 15 days, 20 days, 25 days, 30 days, or 60 days. In some examples, Compound 1, or a pharmaceutically acceptable salt thereof, is administered at least once a day from onset of treatment for a duration of at least about 1 month, 2 months, 3 months, 4 months, 5 months, 6 months, 7, months, 8 months, 9 months, 10 months, or 11 months. In some examples, Compound 1, or a pharmaceutically acceptable salt thereof, is administered at least once a day from onset of treatment for a duration of at least about 1 year. And, in some examples, Compound 1, or a pharmaceutically acceptable salt thereof, is administered at least once a day from onset of treatment for a duration of at least about 2 years, 3 years, 4 years, 5 years, 6 years, 7 years, 8 years, 9 years, or 10 years.

In some implementations, Compound 1, or a pharmaceutically acceptable salt thereof, is administered orally, subcutaneously, intramuscularly, intravenously, intracranially, intrathecally, or intranasally. For example, Compound 1, or a pharmaceutically acceptable salt thereof, is administered orally or intranasally. In some examples, Compound 1, or a pharmaceutically acceptable salt thereof, is administered orally. For example, Compound 1, or a pharmaceutically acceptable salt thereof, is administered as a tablet, capsule, or oral suspension. In some examples, Compound 1, or a pharmaceutically acceptable salt thereof, is administered intranasally. For example, Compound 1, or a pharmaceutically acceptable salt thereof, is administered as a nasal spray.

In some implementations, the subject experiences a vacuolization of mitochondria in GBM cells following treatment onset.

In some implementations, the subject is an animal. For example, the subject is a human.

### IV. EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### A. Materials and Methods

Cell Cultures: Patient-derived glioblastoma (GBM) cell lines isolated post-surgery, commercially available glioma cell lines (87 MG and U-118 MG), breast cancer cell lines (MDA-MB-231 and MCF-7), pancreatic cancer cell lines (MIA PaCa-2 and PANC-1), lung cancer cell lines (H1299), bladder cancer cell lines (T24), acute myeloid leukemia cell lines (NOMO-1, THP-1), colon cancer cell lines (HT-29), neuroblastoma cell lines (BE(2)-C), and human foreskin fibroblasts (CRL-2429) were grown as adherent cultures in Dulbecco Modified Eagles Medium (DMEM) (GE Healthcare Life Sciences), 10% Fetal Bovine Serum (FBS) (GE Healthcare Life Sciences) and 1× Penicillin-Streptomycin (Penstrep) (GE Healthcare Life Sciences). Cell cultures were split upon 90% confluence.

Cell Viability Assay: Cell viability assays were performed by measuring the total adenosine triphosphate (ATP) levels of live cells (CellTiter-Glo® assay (Promega) according to the manufacturer's protocols). Luminescence was measured using a Victor3 FA (PerkinElmer) microtiterplate reader and plotted as graphs using GraphPad Prism (v6.02) software.

Primary Screening: Primary screening was performed using cells plated in triplicate in a 96 well plate (3,000 cells per well) and left to adhere for at least 3 hours. Media was changed to DMEM (without FBS+10% Penstrep), and the cells were left to acclimate for 45 minutes prior to compound addition. Compounds were added to a final concentration of 10 UM and the cells were incubated at 37° C. for four days. ATP levels were measured after incubation.

IC₅₀ Measurements: To determine compound dose-response inhibition of cell viability, a 96-well polypropylene (PP) microtiter compound plate (Nunc) was prepared to give 20 L/well of a continuous 11-point dose-response dilution from 500 μM-500 nM compound in 100% DMSO in columns 1-9 of each row. Negative (100% DMSO) and positive (10 mM Staurosporine in DMSO) controls were placed in columns 10 and 11, respectively. The plate was diluted with 180 μL growth media/well and 5 μL of the resulting compound solution was transferred in quadruplicate to the assay plate. For the assay plate, 3,000 cells/well were plated in a 384 well clear bottom μL plate and allowed to adhere for at least 3 hours. Then, the media was changed to DMEM (without FBS+10% Penstrep), and the cells were left to acclimate for 45 minutes prior addition of 5 μL of the compound from the compound plate. The plates were incubated for 1, 2, 3, or 4 days. ATP levels were measured after incubation.

Long-term Exposure Assay: Measurements for long-term assays were performed on days 4, 8, 14, and 21. 3,000 cells/well were plated in a 384 well plate (for day 4 assay), a 96 well plate (for day 8 assay), a 24 well plate (for day 14 assay), a 6 well plate (for day 21 assay), or a 10 cm plate (for day 30 assay) and allowed to adhere as above. Following this, media was exchanged to DMEM (without FBS+1× Penstrep) with either DMSO as control treatment or compound (at final concentration of 0.1 μM or 5 μM). Every four days, media was replaced with fresh DMEM (without FBS+1×Penstrep) comprising DMSO (control) or compound. At the end of days 4, 8, 14, and 21 cell viability assays were performed.

Transfection using siRNA: Cells were plated in triplicate at 3,000 cells/well and treated with 40 or 50 nmol siRNA against SLC6A3 (siGENOME Human SLC6A3 (6531 siRNA SMARTpool, 5 nmol (Dharmacon)) or 40 nmol Scramble (siGENOME Non-Targeting siRNA Pool #1, 20 nmol (Dharmacon) as internal control, in DMEM (-FBS, -Penstrep). Total siRNA/HiPerfect® Transfection Reagent 29 (QIAGEN) prepared in 16% of total volume, siRNA prepared in OPTIMEM (8% of total volume), incubated for 5 minutes at room temperature (RT) and mixed with HiPerfect/OPTIMEM solution (8% of total volume with 0.3% HiPerfect) and incubated for 20 minutes at RT. After 20 hours, media was changed to DMEM (10% Penstrep), and the compound (at concentrations used in the IC₅₀ studies) was added and incubated for 48 hours and a cell viability assay was performed. To determine the knock down efficiency, total RNA from cell treated with scrambled or siRNA was isolated using TRIzol method (as per manufacturer's instruction) and cDNA was prepared using SuperScript III First-Strand Synthesis SuperMix. Using cDNA, qPCR was performed with SYBR Select Master Mix (Applied Biosystems) and primers created with Primer3 software towards SLC6A3 (500 nM) (primer pair SLC6A3:1 forward: TCACCAACGGTGGCATCTAC (SEQ ID NO: 1), reverse; TCATCTGCTGGATGTCGTCG (SEQ ID NO: 2), 5′ to 3′ generating a 144 bp amplicon (Integrated DNA technologies) and SLC6A3:2 forward; TCACCAACGGTGGCATCTAC (SEQ ID NO: 3), reverse; CACTCCGATGGCTTCGATGA (SEQ ID NO: 4) 5′ to 3′, generating a 95 bp amplicon (Schrödter et al, 2016) (Integrated DNA technologies)); Beta actin control (500 nM) (Hs_ACTB_2_SG QuantiTect Primer Assay (Qiagen)). PCR conditions were 50° C. for 2 minutes (UDG activation), 95° C. for 2 minutes (AmpliTaq DNA pol activation) and 40 cycles of 95° C. for 30 seconds, 60° C. for 30 seconds and 72° C. for 60 seconds on a Corbette research, RotorGene 6000 series software 1.7. Housekeeping genes were run in concentration 1:2.5, 1:5, 1:25, while SLC6A3 were run on 1:5 and 1:25 (from 1000 ng/μL cDNA). Samples were pooled and run on Lonza Flash Gel Cassette (10 μL, 0.5 mg/μL, 1.2% 12+1 Single Tier DNA (Lonza)) at 4° C. Analysis of the obtained results were performed by relative expression with the ΔΔCt method.

### Example 1: Preliminary Cytotoxicity Studies

The cytotoxic effect of various compounds was examined in the patient-derived cell lines described in the Material and Methods above. The compounds were categorized as antipsychotics, stimulants, anticonvulsants and mood stabilizers, antidepressants and benzodiazepines, sedatives, and hypnotics based on their previously established activity.

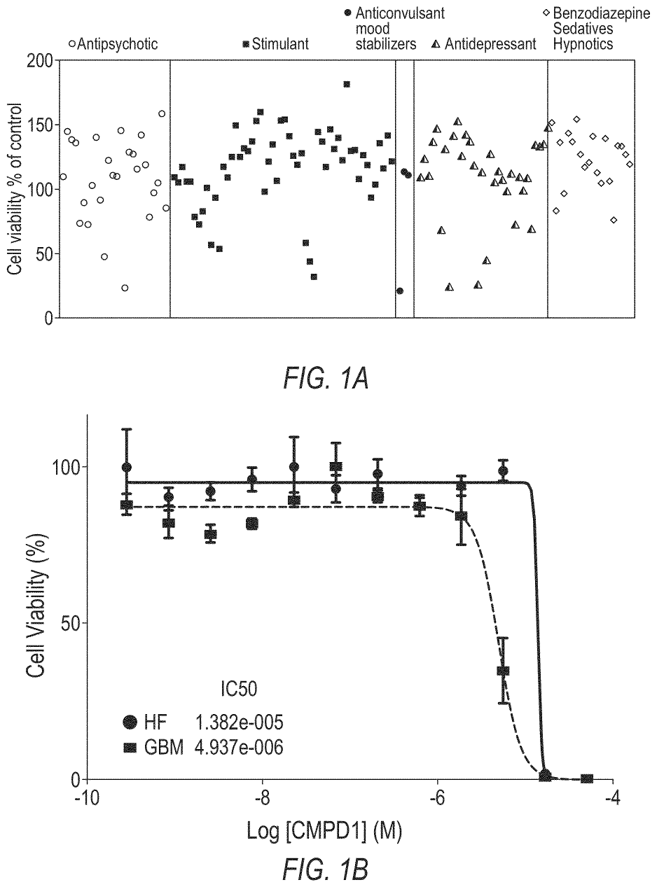

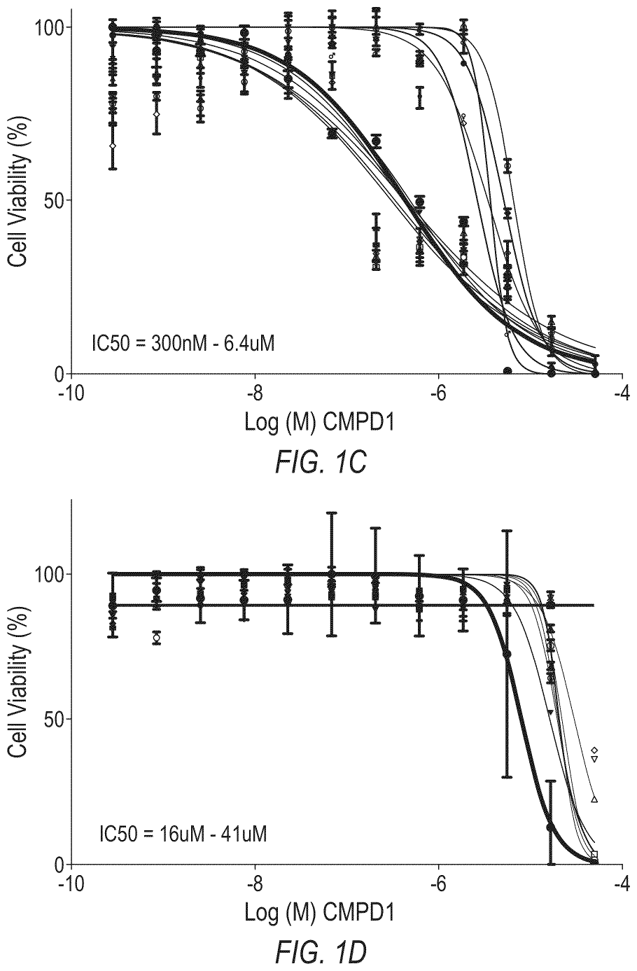

Referring to FIG. 1A, general low toxicity was detected after treatment with benzodiazepines, sedatives, and hypnotics as compared to the other categories of compounds. The most effective compounds were found to be anticonvulsants, mood stabilizers, antidepressants, antipsychotics, and stimulants that targeted the dopaminergic system.

All compounds targeting the dopaminergic pathway were further categorized based on their target receptor and analyzed in brain, breast, colon, lung, and pancreatic cancer cell lines. The results are shown in Tables 1-3. The cancer cells were isolated from GBM, breast cancer, colon cancer, pancreatic cancer, and Lung cancer tumors. The compounds tested included dopamine agonists (DAg), dopamine (DA), dopamine reuptake inhibitors (DRI), dopamine uptake inhibitors (DUI), and dopamine antagonists (DAn). DMSO was used as control and was assigned value of 1. Activity of each compound was compared to DMSO and categorized. The compounds tested are provided in Tables 1-3. The most cytotoxic compounds for a particular cancer cell line are categorized with an “A” and the least cytotoxic compounds for a particular cancer cell line are categorized with a “D”. Compounds exhibiting intermediate cytotoxicity are categorized with a “B” or a “C”. In Tables 1-3, “A” indicates a score of less than 0.60 (i.e., A<0.60), “B” indicates a score of from greater than or equal to 0.60 to less than 1.00 (i.e., 0.60≤B<1.00), “C” indicates a score of from greater than or equal to 1.00 to less than 1.30 (i.e., 1.00≤C<1.30), and “D” indicates a score of greater than or equal to 1.30 (i.e., D≥1.30).

TABLE 1
Cytotoxicity of various dopamine antagonists towards certain cancer cell lines.

| | Brain | | | | |
|---|---|---|---|---|---|
| | Cancer | Breast | | Pancreatic | |
| | (Patient 1 | Cancer | Colon | Cancer | Lung |
| Dopamine | GBM | (MDA- | Cancer | (MIA- | Cancer |
| Antagonist | cells) | MB-231) | (HT-29) | PACA) | (H1299) |
| L750,667 3HCl | A | C | A | C | A |
| GR 103691 | A | A | D | C | B |
| 3′- | A | B | D | B | A |
| Fluorobenzylspiperone | | | | | |
| maleate | | | | | |
| Clothiapine | A | B | B | C | B |
| AJ-76 | B | B | C | C | B |
| Thioridazine HCl | B | A | A | B | A |
| SCH 23390 | B | B | B | C | C |
| Cis-(Z)-flupenthixol | B | C | A | B | B |
| 2HCl | | | | | |
| Fluphenazine 2HCl | B | A | A | B | A |
| Trifluoperazine 2HCl | B | B | A | B | A |
| 2-Chloro-11-(4- | B | B | D | C | A |
| methylpiperazino) | | | | | |
| dibenz[B,F]oxepin | | | | | |
| Maleate | | | | | |
| Clozapine | B | C | A | C | B |
| U-99194A maleate | B | B | C | C | B |
| (+)-Butaclamol | B | C | A | C | B |
| L-741,742 HCl | B | B | D | B | A |
| L-741,626 | B | C | A | B | A |
| Risperidone | B | B | C | C | B |
| S(−)-Lisuride | B | B | B | B | A |
| Ergonovine maleate | B | B | B | C | B |
| S(−)-Sulpiride | B | C | C | C | B |
| Trans-7-hydroxy-pipat | B | B | A | B | A |
| maleate | | | | | |
| Haloperidol HCl | C | B | A | B | A |
| DMSO | 1.00 | 1.00 | 1.00 | 1.00 | 1.00 |
| (Control) | | | | | |
| Metoclopramide HCl | C | C | A | B | A |
| Mesoridazine besylate | C | C | B | C | B |
| 7-Hydroxy-pipat | C | C | D | C | B |
| maleate | | | | | |
| R(+)-Terguride | C | C | B | C | A |
| (S)-(−)-Sulpiride | C | C | D | C | C |
| (RS)-(±)-Sulpiride | C | C | C | C | B |
| Domperidone | C | C | A | C | B |
| S-(−)-Raclopride L- | C | C | C | C | D |
| tartrate | | | | | |
| L-741,741 HCl | C | B | C | B | A |
| (±)-SKF-83566 HCl | C | C | B | C | B |
| L-745,870 3 HCl | C | C | B | C | B |
| N-methyldopamine | C | B | B | C | B |
| HCl | | | | | |
| Dihydroergocristine | C | C | A | C | A |
| mesylate | | | | | |
| Remoxipride | D | C | C | C | C |
| AMI-193 | D | A | D | C | A |

TABLE 2
Cytotoxicity of various dopamine agonists towards certain cancer cell lines.

| | Brain | | | | |
|---|---|---|---|---|---|
| | Cancer | Breast | | Pancreatic | |
| | (Patient 1 | Cancer | Colon | Cancer | Lung |
| Dopamine | GBM | (MDA- | Cancer | (MIA- | Cancer |
| Agonist | cells) | MB-2321) | (HT-29) | PACA) | (H1299) |
| L750,667 3HCl | A | C | A | C | A |
| R-(+)-6-bromo-APB | B | B | B | C | A |
| HBr | | | | | |
| Bromocriptine mesylate | B | C | B | C | B |
| R-(+)-SKF-81297 | B | B | B | C | B |
| (+)-PD 128907 HCl | B | C | D | C | B |
| (±)-SKF-82958 HBr | B | B | A | C | A |
| (±)-2-(N-phenylethyl-N- | B | C | A | C | A |
| propyl)amino-5- | | | | | |
| hydroxytetralin | | | | | |
| Dihydrexidine HCl | B | B | A | C | A |
| R-(−)-Apomorphine | B | B | C | C | B |
| 4- | B | C | B | C | A |
| Hydroxyphenethylamine | | | | | |
| HCl | | | | | |
| (−)-Quinpirole HCl | B | C | C | C | D |
| L-741,742 HCl | B | B | D | B | A |
| S-(−)-Lisuride | B | B | B | B | A |
| Mesulergine HCl | B | C | B | C | B |
| (+)-UH 232 Maleate | B | C | D | C | A |
| 3- | B | C | B | C | B |
| Hydroxyphenethylamine | | | | | |
| HCl | | | | | |
| R-(−)- | B | B | A | C | A |
| Propylnorapomorphine | | | | | |
| HCl | | | | | |
| Ergonovine maleate | B | B | B | C | B |
| Chlorpromazine HCl | B | B | A | C | A |
| DMSO | 1.00 | 1.00 | 1.00 | 1.00 | 1.00 |
| (Control) | | | | | |
| 7-Hydroxy-DPAT HBr | C | C | B | B | A |
| Ergocornine | C | C | C | C | C |
| R-(−)-N- | C | B | A | C | A |
| Allylnorapomorphine | | | | | |
| HBr | | | | | |
| GBR 12783 2HCl | C | B | B | C | B |
| A-77636 HCl | C | C | A | C | B |
| R-(−)-2,10,11- | C | C | B | C | A |
| trihydroxy-N-propyl- | | | | | |
| noraporphine HBr | | | | | |
| R-(−)-2,11-dihydroxy- | C | B | C | C | A |
| 10-methoxyaporphine | | | | | |
| HCl | | | | | |
| PD 168077 maleate | C | C | C | C | C |
| CY 208-243 | C | C | D | C | B |
| R-(+)-Terguride | C | C | B | C | A |
| LY-163,502 | C | C | C | C | D |
| Agroclavine | C | C | B | C | A |
| Pergolide | C | C | B | C | C |
| methanesulfonate | | | | | |
| Dopamine HCl | C | B | B | C | B |
| SKF 38393 HBr | C | C | B | C | B |
| N-Methyldopamine HCl | C | B | B | C | B |
| 6,7-ADTN HBr | C | B | C | C | B |
| Alpha-ergocryptine | C | C | C | C | B |
| Dihydroergocristine | C | C | A | C | A |
| mesylate | | | | | |
| Dipropyldopamine HBr | C | B | B | C | A |
| R-(−)-2,10,11- | D | B | A | A | A |
| tryihydroxyaporphine | | | | | |
| HBr | | | | | |

TABLE 3
Cytotoxicity of various dopamine release inhibitors, dopamine uptake inhibitors,
and dopamine reuptake inhibitors towards certain cancer cell lines.

| | Brain | | | | |
|---|---|---|---|---|---|
| | Cancer | Breast | | Pancreatic | |
| Dopamine | (Patient 1 | Cancer | Colon | Cancer | Lung |
| Release/Uptake/ | GBM | (MDA- | Cancer | (MIA- | Cancer |
| Reuptake Inhibitor | cells) | MB-231) | (HT-29) | PACA) | (H1299) |
| GBR-12909 | A | A | A | A | A |
| (i.e., Compound 1) | | | | | |
| 1-[1-(2- | A | B | B | C | B |
| Benzo[B]thienyl) | | | | | |
| cyclohexyl]pyrrolidine | | | | | |
| fumarate | | | | | |
| 4-Phenyl-1,2,3,4- | C | B | B | C | A |
| tetrahydroisoquinoline | | | | | |
| HCl | | | | | |

The results demonstrate that various compounds known to be dopamine antagonists, dopamine agonists, and dopamine release/uptake/reuptake inhibitors exhibit significant cytotoxicity towards particular cancer cell lines, relative to the cytotoxicity of DMSO. Of these compounds, Compound 1 (i.e., GBR-12909 in Table 3) exhibited the most potent level of cytotoxic activity (i.e., level A) towards all tested cell lines.

### Example 2: Additional Cytotoxicity Studies

Based on the results of Example 1, Compound 1 was selected for further evaluation. The half maximal inhibitory concentration (IC₅₀) of Compound 1 was obtained after 4 days of treatment based on a cell viability assay and summarized in Table 4, below.

TABLE 4
IC₅₀ values for Compound 1 in certain cancer cell lines.

| | Mean IC₅₀ (μM) | | | | |
|---|---|---|---|---|---|
| | Brain | | | | |
| | Cancer | Breast | | Pancreatic | |
| | (Patient 1 | Cancer | Colon | Cancer | Lung |
| | GBM | (MDA- | Cancer | (MIA- | Cancer |
| Compound | cells) | MB-231) | (HT-29) | PACA) | (H1299) |
| Compound 1 | 2.2 | 2.3 | 2.4 | 2.4 | 2.1 |

As shown in Tables 3 and 4, Compound 1 exhibited uniformly strong cytotoxic activity in all the cancer cell lines studied. In this initial study, the selectivity of Compound 1 for inhibiting cancer cells as compared with normal cells was not evaluated.

### Example 3: Compound 1 in GBM Cells

FIG. 1B shows serial dilution experiments of Compound 1 were performed in patient-derived GBM cells and fibroblasts isolated from the same patient after 4 days of incubation. The serial dilution studies showed an IC₅₀ of 4.9 μM for glioma cells from patient 1 and 13.8 μM for fibroblast cells from patient 1. FIGS. 1C and 1D show serial dilution experiments performed multiple times on various cell passages in order to determine the variability in IC₅₀ values. Referring to FIG. 1C, for GBM cells, the IC₅₀ of Compound 1 was found to be from about 300 nM to about 6.4 μM. Referring to FIG. 1D, for the fibroblasts, the IC₅₀ of Compound 1 was found to be from about 16 μM to about 41 μM.

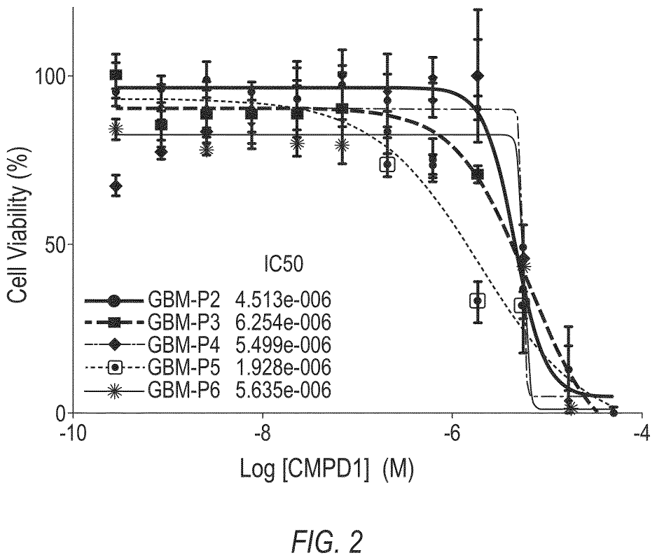

Serial dilutions in FIG. 2 show the effect of Compound 1 on GBM cells isolated from an additional five patients. An IC₅₀ average of 4.72 μM was observed for GBM cells. This indicates that Compound 1 acted as a potent cytotoxin toward glioma cells and demonstrates the potential utility of Compound 1 as a therapeutic agent.

### Example 4: Long-Term Exposure Studies of Compound 1 in GBM Cells

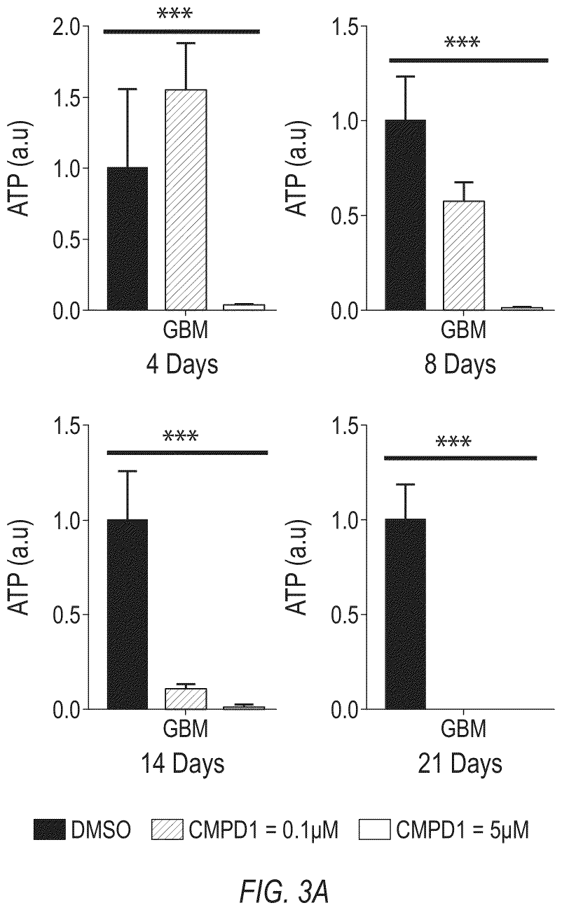

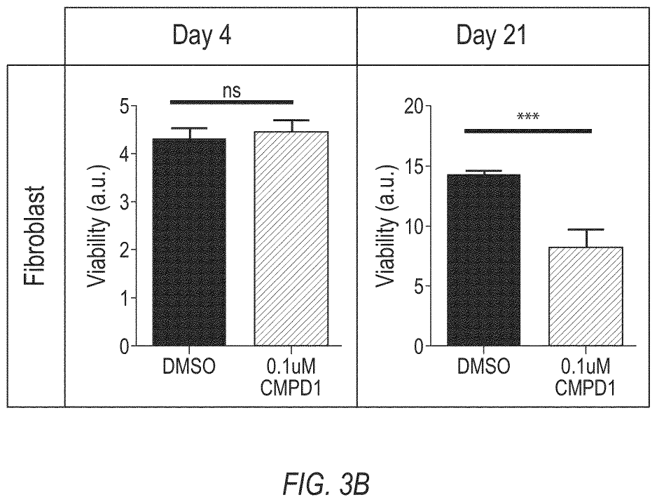

Patient-derived GBM cells were treated with Compound 1 at concentrations of 5 μM or 0.1 μM and incubated for 21 days. Fibroblasts were used as a control (FIG. 3B). As shown in FIG. 3A, Compound 1 at 0.1 μM eliminated the GBM cells without exhibiting cytotoxicity towards the fibroblasts (i.e., the control). These results indicate that Compound 1 has the potential to be administered at therapeutically effective doses to treat GBM without affecting non-GBM cells.

### Example 5: Dopamine Transporter Knockdown and Cell Death Analysis in GBM Cells

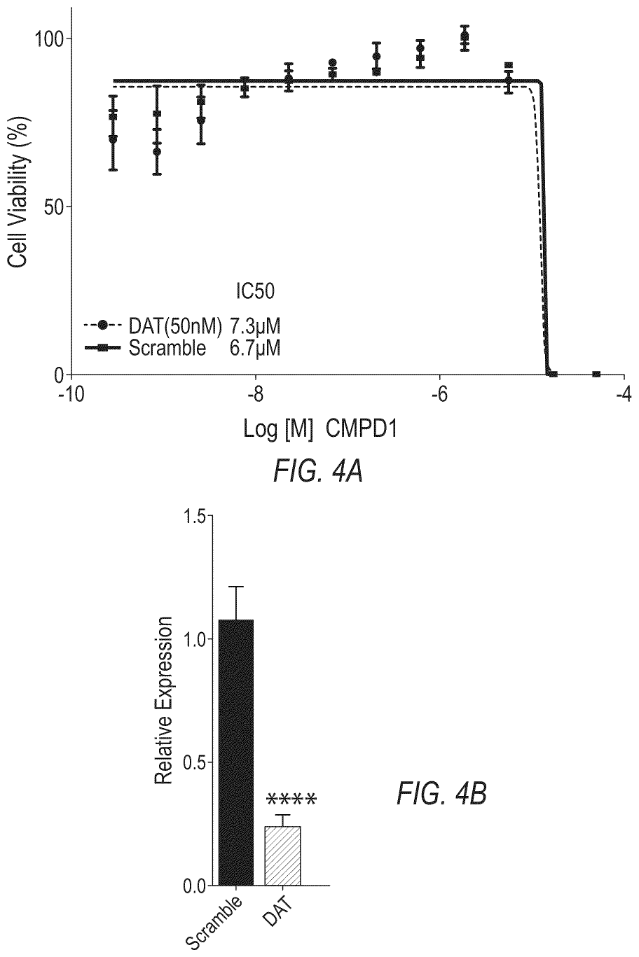

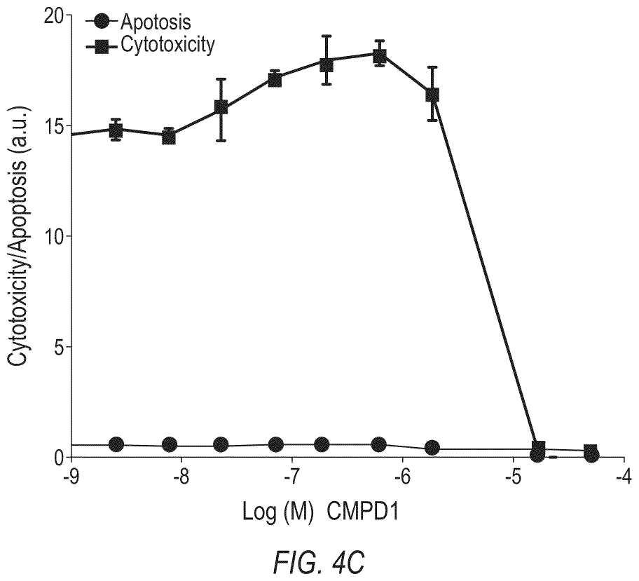

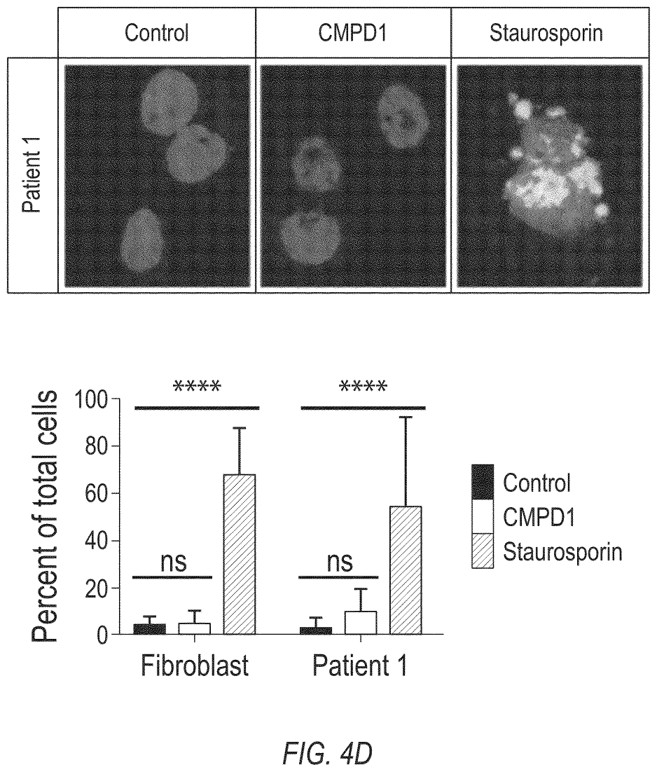

Compound 1 is known to bind the dopamine transporter (DAT) and also inhibit the release of dopamine. In order to evaluate whether the cytotoxicity of Compound 1 in GBM cells is attributable to its inhibition of dopamine reuptake, DAT was knocked down in GBM cells using siRNA. Scrambled samples were used as controls. As shown in FIG. 4A, Compound 1 exhibited similar IC₅₀ values in both the DAT-knockdown cells and the control cells (scrambled) after 96 hours of treatment while the relative expression of DAT is dramatically reduced in the cells treated with siRNA against DAT. If the cytotoxic activity of Compound 1 were attributable to its modulation of DAT, then Compound 1 would not be cytotoxic towards the DAT-knockdown cells. As such, these results suggest that the cytotoxic activity of Compound 1 is likely attributable to a target other than DAT.

As shown in FIG. 4B, analysis of cytotoxicity and apoptosis assays (performed using ApoTox-Glo (Promega) kit as per the manufacturer's instructions) indicates that cells treated with Compound 1 produced immense cytotoxicity but apoptosis was not initiated in Glioma cells. As shown in FIGS. 4C and 4D, immunostaining with Caspase 3 antibody (an apoptosis marker) indicated that Compound 1 did not produce apoptosis positive cells (similar to control) when compared to the positive control, staurosporin, for apoptosis in glioma cells from patient 1. The immunostaining followed by quantification of Annexin V positive cells indicates that Compound 1 treated cells do not produce more apoptosis positive cells in fibroblast control and glioma cells from patient 1. These results suggest that Compound 1 does not act via DAT receptor and the dopaminergic pathway, and does not give rise to cell death via apoptosis.

### Examples 6 and 7: Vacuolization and Depolarization of Mitochondria in GBM Cells Treated with Compound 1

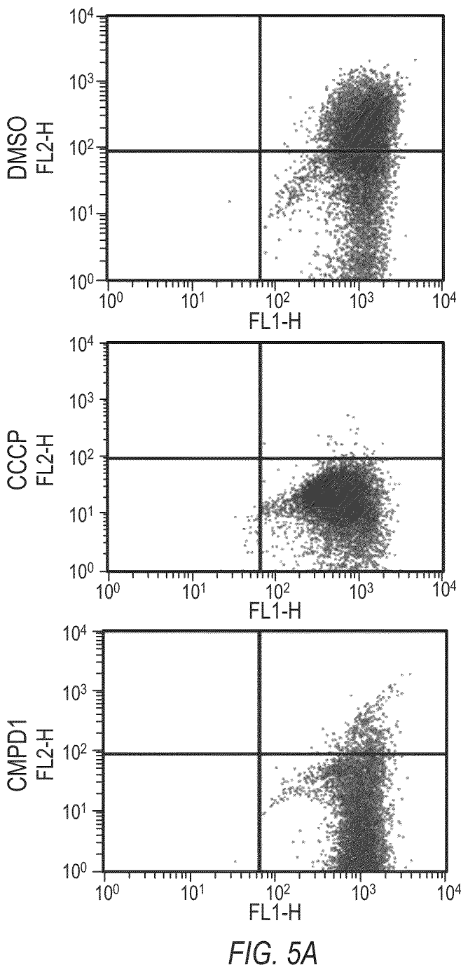

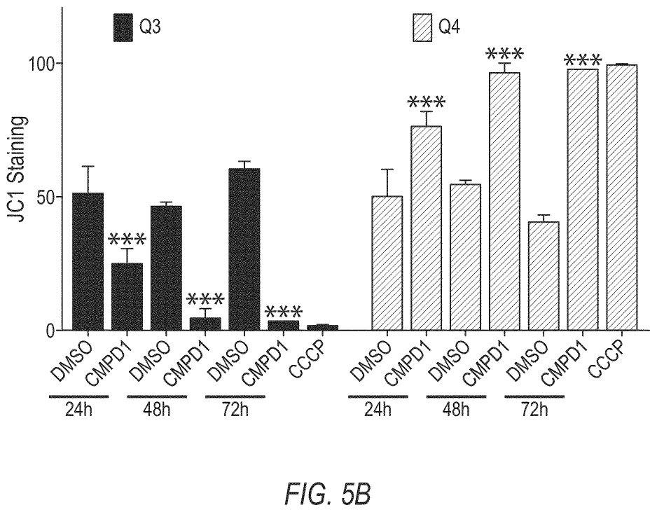

GBM cells were treated with Compound 1, carbonyl cyanide m-chlorophenyl hydrazine (CCCP; positive control), or DMSO (negative control) and the mitochondrial membrane potential (indicative of functional status of the GBM cells) was measured and analyzed. With reference to FIGS. 5A and 5B, treatment with Compound 1 resulted in a depolarization of mitochondria in a manner similar to CCCP (i.e., the positive control).

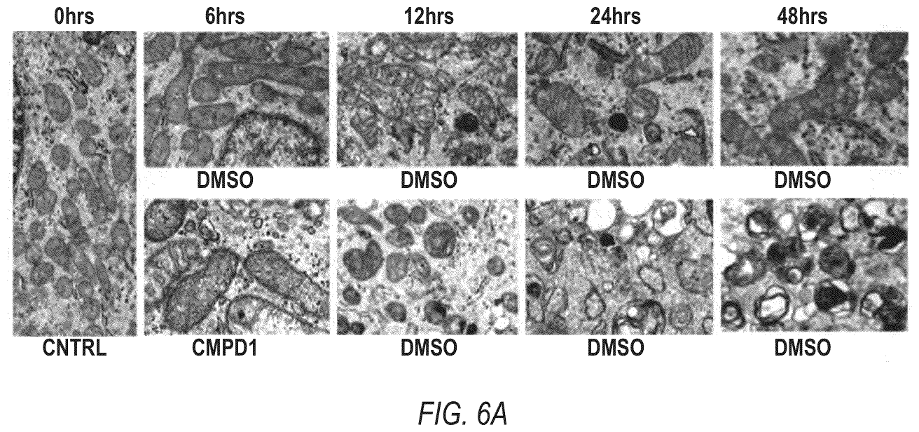

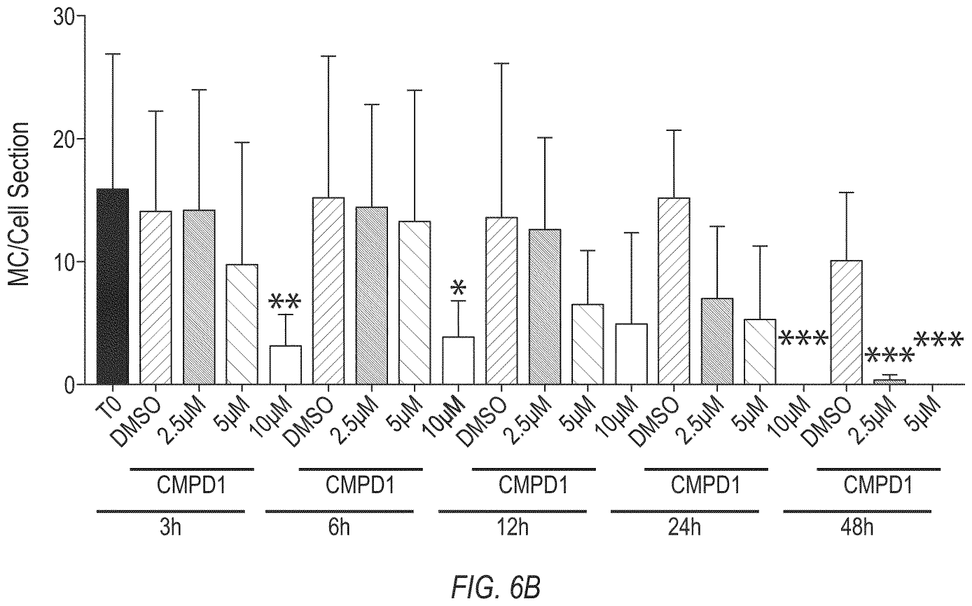

Separately, GBM cells were treated with Compound 1 (2.5 μM, 5 μM, or 10 μM) or DMSO (control) and incubated for 48 hours. Referring to FIGS. 6A and 6B, the GBM cells were analyzed via Transmission Electron (TEM) Microscopy after 3, 6, 12, 24, and 48 hours of incubation. Treatment with Compound 1 induced vacuolization of mitochondria in the GBM cells.

### Example 8: Identification of Potential Targets for Compound 1 in GBM Cells

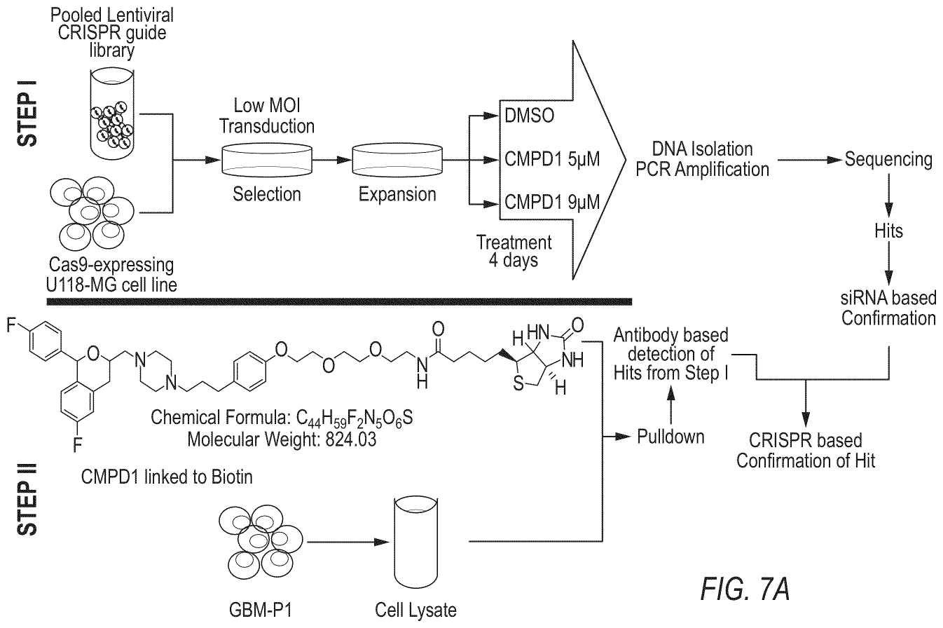

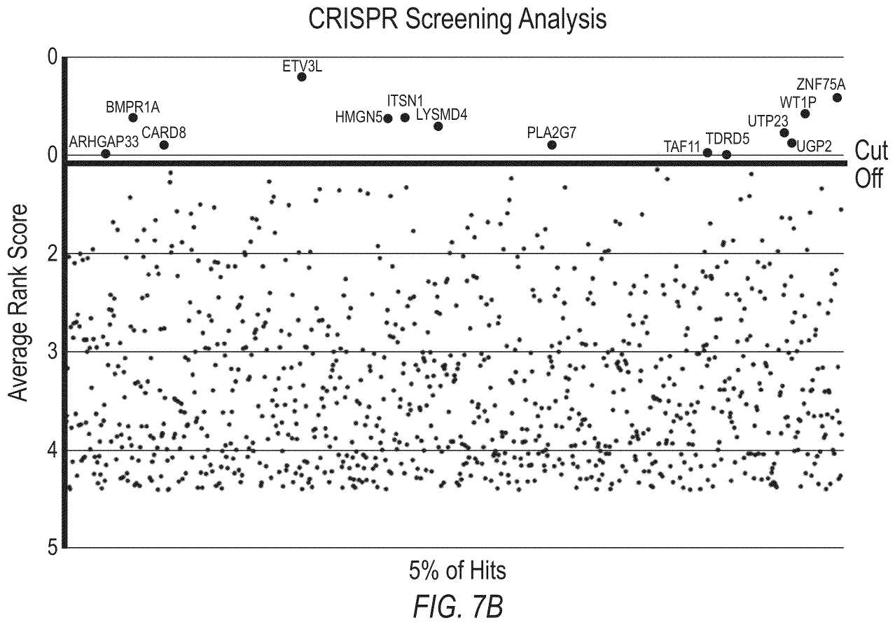

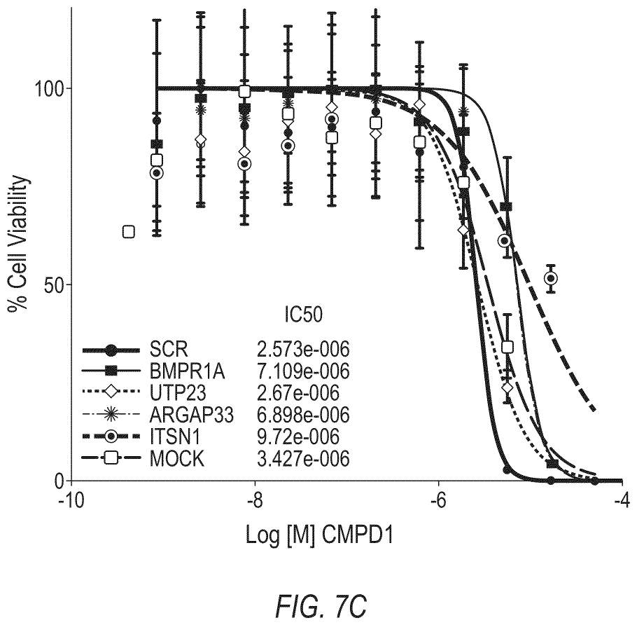

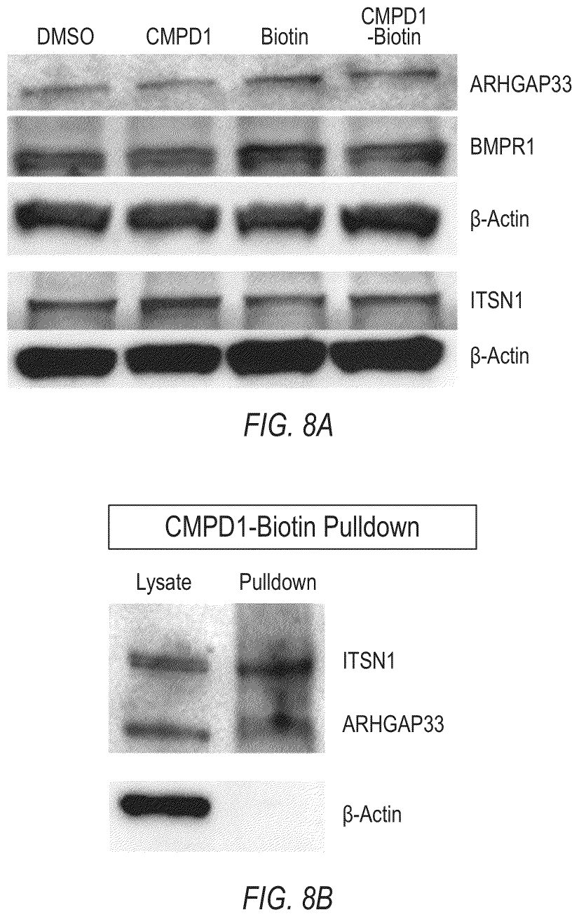

With reference to Step I in FIGS. 7A and 7B, whole genome CRISPR-Cas9 studies (B. Schmierer, Mol Systems Biol., 13, 945 (2017)) were carried out to identify potential targets that could explain the effect produced by Compound 1 on cancer cells. The top 5% of the targets shown in FIG. 7B were identified and some of them were confirmed by siRNA based knockdown either with controls (mock-without siRNA or scrambled control siRNA (SCR)) or gene specific siRNA (for BMPR1A, UTP23, ARHGAP33, ITSN1) as seen in FIG. 7C, followed by treatment with or without Compound 1. With reference to FIG. 7C, knockdown of targets BMPR1A, ARHGAP33 and ITSN1 resulted in higher IC₅₀ values when treated with Compound 1, indicating some resistance to the cytotoxic effect of Compound 1. This indicates that these targets are required for maximal cytotoxic activity of Compound 1. With reference to Step II in FIG. 7A and FIGS. 8A and 8B, after the CRISPR screening analysis was complete, the expression of identified hits were confirmed in GB cells as shown in FIG. 8A. The binding of Compound 1 to the identified hits was established via a pull-down assay using biotin labeled Compound 1 (FIG. 8B). The results indicate that the observed cytotoxic effect of Compound 1 in GBM cells may be related to modulation of BMPR1A, UTP23, ARHGAP33, and/or ITSN1

### Example 9: Effect of Compound 1 in Mouse Models

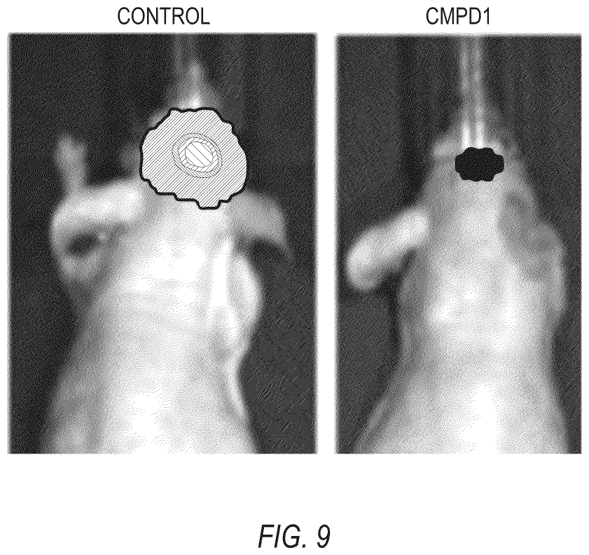

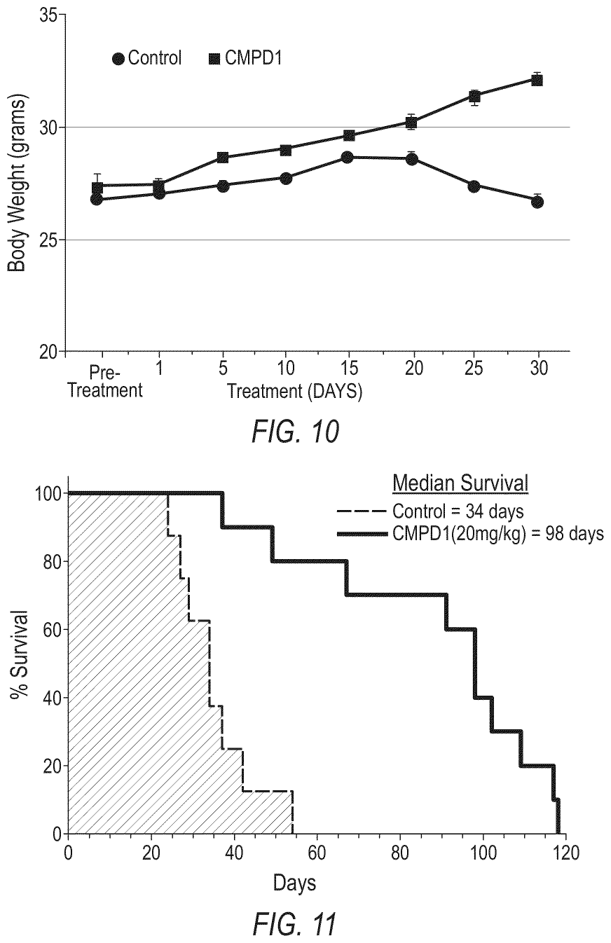

A GBM cancer cell line was xenografted into 8-week old male nude mice and the tumor was allowed to develop for four weeks. The GBM cells were stereotactically implanted into the brain and allowed to grow for four weeks. After four weeks, treatment with Compound 1 via oral administration was started (Day 1). The mice were allowed to live until the ethical endpoint of the study was reached. Body weight and activity of animals were monitored continuously. As shown in FIG. 9, mice treated with Compound 1 exhibited a reduction in tumor size as compared to mice that were not treated with Compound 1. As shown in FIG. 10, mice treated with Compound 1 experienced a reduced loss in bodyweight as compared to mice that were not treated with Compound 1. As shown in FIG. 11, mice treated with Compound 1 had an increased life span as compared to mice that were not treated with Compound 1. Mice treated with Compound 1 also displayed increased physical activity as compared to mice that were not treated with Compound 1. These results indicate that Compound 1 may be useful in both treating GBM and also in providing palliative care to subjects afflicted with GBM.

### Example 10: Combination Therapy Studies

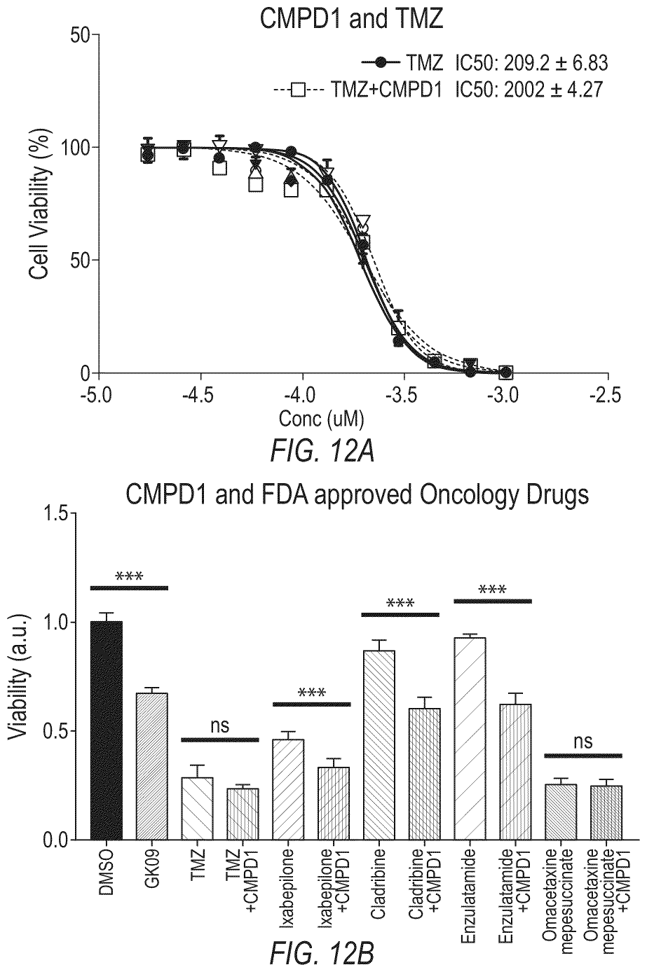

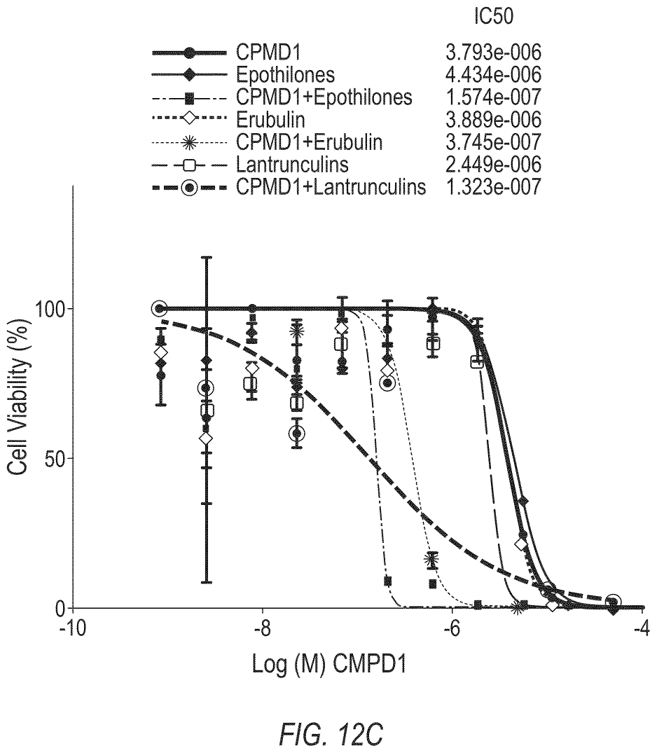

GBM cells were treated with Compound 1 and various other U.S. Food and Drug Administration (FDA) approved oncology drugs. The FDA approved oncology drugs included temozolomide (TMZ), ixabepilone, cladribine, enzalutamide, omacetaxine mepesuccinate, epothilones, erubulin, and latrunculin. Treatment by Compound 1 in the additional presence of ixabepilone, cladribine, or enzalutamide resulted in statistically significant decreases in cell viability. With reference to FIGS. 12A-C, the results indicated that Compound 1 may be effectively combined with other FDA approved oncology drugs to treat GBM.

These Examples demonstrate the potential of Compound 1, a selective dopamine uptake inhibitor, as a broad spectrum cytotoxic agent. Compound 1 was more potent and broadly acting as a cytotoxic agent than any of a number of dopamine antagonists, dopamine agonists, and dopamine release/uptake/reuptake inhibitors, as documented in Examples 1 and 2. In Examples 3 and 4, it is shown (FIGS. 1B-3B) that Compound 1 inhibits the growth of GBM cells, and does so to a greater extent than it inhibits human fibroblasts. In Example 5, it is shown that Compound 1 does not seem to exert its anti-gliobastoma effects through the dopamine transporter. Instead, Compound 1 appears to act through the vacuolization and depolarization of mitochondria in GBM cells (Example 7). Using a genomic CRISPR-Cas9 screen, it was shown that the inhibition may be related to modulation of BMPR1A, UTP23, ARHGAP33, and/or ITSN1 (Example 8). Compound 1 was also shown to inhibit the growth of GBM cells introduced into mice as a xenograft. In addition to a reduction in tumor size as a consequence of administration of Compound 1, the treated mice also displayed increased physical activity as compared to mice that were not treated with Compound 1, and lost weight to a lesser extent than xenograft-bearing mice not treated with Compound 1 (Example 9). In studies involving the use of other FDA-approved oncology drugs, the additional presence of ixabepilone, cladribine, or enzalutamide resulted in statistically significant increases in cell viability (Example 10). Compound 1 has previously been tested in clinical trials as a dopamine uptake inhibitor, establishing its safety when administered to humans.

The foregoing data supports the potential utility of Compound 1 for the treatment of gliomas, and suggests that Compound 1 operates by a unique mechanism of action involving inhibition of glioblastoma cell mitochondria. Thus, while other compounds have been tested for the therapy of gliomas, as exemplified by recent work involving CDK inhibitors (Bronner et al. Bioorg. Med Chem., 29, 2294 (2019); Yin et al. Eur. J. Med Chem., 144, 1, (2018)), these compounds appear to operate as kinase inhibitors and not via inhibition of mitochondria.

As demonstrated in Example 10, the activity of Compound 1 can be augmented by at least three FDA-approved anticancer agents. This finding suggests that Compound 1 may be suitable as a single agent, or in therapeutic regimens, for treatment of cancers other than glioblastomas, as does its broad cytotoxic activity against a variety of cancer cell types (Examples 1 and 2).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating cancer in a subject in need thereof, comprising administering to the subject a therapeutically effective amount of Compound 1 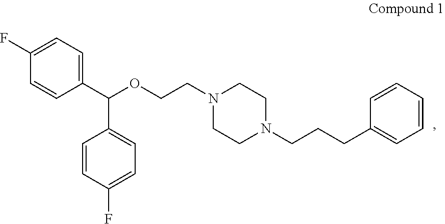 [SMILES: Fc1ccc(C(OCCN2CCN(CCCc3ccccc3)CC2)c2ccc(F)cc2)cc1]
or a pharmaceutically acceptable salt thereof, wherein the cancer is selected from brain cancer, breast cancer, pancreatic cancer, lung cancer, or any combination thereof.

2-22. (canceled)

23. A method of treating glioma in a subject in need thereof, comprising administering to the subject a therapeutically effective amount of Compound 1 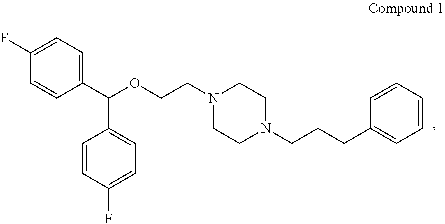 [SMILES: Fc1ccc(C(OCCN2CCN(CCCc3ccccc3)CC2)c2ccc(F)cc2)cc1]
or a pharmaceutically acceptable salt thereof.

24. The method of claim 23, wherein Compound 1, or the pharmaceutically acceptable salt thereof, is a hydrochloride salt of Compound 1.

25. The method of claim 23, wherein Compound 1, or the pharmaceutically acceptable salt thereof, is a dihydrochloride salt of Compound 1.

26. The method of claim 23, wherein the glioma is selected from the group consisting of an astrocytoma, an ependymoma, an oligodendroglioma, a brainstem glioma, an optic nerve glioma, and a mixed glioma.

27. The method of claim 23, wherein the glioma is an astrocytoma, and wherein the astrocytoma is glioblastoma multiforme (GBM).

28. The method of claim 23, wherein Compound 1, or the pharmaceutically acceptable salt thereof, modulates activity of BMPR1A, UTP23, ARHGAP33, ITSN1, or any combination thereof in glioma cells.

29. The method of claim 23, wherein Compound 1, or the pharmaceutically acceptable salt thereof, is administered as a pharmaceutical composition additionally comprising a pharmaceutically acceptable carrier, excipient, or diluent.

30. The method of claim 23, wherein Compound 1, or the pharmaceutically acceptable salt thereof, is administered at least once per day.

31. The method of claim 23, further comprising administering to the subject an additional therapeutic agent, wherein the additional therapeutic agent is an anti-cancer agent selected from temozolomide, ixabepilone, cladribine, enzalutamide, omacetaxine mepesuccinate, epothilones, erubulin, latrunculin, a pharmaceutically acceptable salt of any of these anti-cancer agents, or any combination thereof.

32. (canceled)

33. (canceled)

34. (canceled)

35. (canceled)

36. The method of claim 23, further comprising administering to the subject a radiation therapy.

37. The method of claim 23, wherein Compound 1, or the pharmaceutically acceptable salt thereof, is administered in a dosage amount of from about 1 mg to about 1,000 mg.

38. The method of claim 23, wherein Compound 1, or the pharmaceutically acceptable salt thereof, is administered chronically.

39. The method of claim 23, wherein Compound 1, or the pharmaceutically acceptable salt thereof, is administered orally, subcutaneously, intramuscularly, intravenously, intracranially, intrathecally, or intranasally.

40. The method of claim 23, wherein Compound 1, or the pharmaceutically acceptable salt thereof, is administered orally.

41. The method of claim 23, wherein Compound 1, or the pharmaceutically acceptable salt thereof, is administered as a tablet, capsule, or oral suspension.

42. The method of claim 23, wherein Compound 1, or the pharmaceutically acceptable salt thereof, is administered at least once a day from onset of treatment for a duration of at least about one year.

43. The method of claim 23, wherein the subject experiences at least one of:
a reduced loss in bodyweight following treatment onset as compared to a subject not administered Compound 1, or a pharmaceutically acceptable salt thereof;
a vacuolization of mitochondria in glioma cells following treatment onset; and
a depolarization of mitochondria in cancer cells following treatment onset.

44. (canceled)

45. (canceled)

46. A method of
inducing vacuolization of mitochondria in glioblastoma multiforme (GBM) cells; or
depolarizing mitochondria in GBM cells, of a subject, the method comprising administering to the subject a therapeutically effective amount of Compound 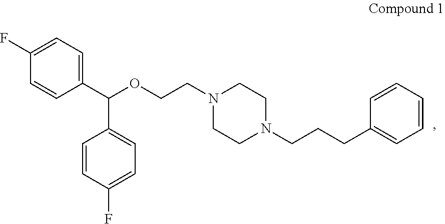 [SMILES: Fc1ccc(C(OCCN2CCN(CCCc3ccccc3)CC2)c2ccc(F)cc2)cc1]
or a pharmaceutically acceptable salt thereof.

47-75. (canceled)

76. The method of claim 29, wherein the pharmaceutical composition is a nasal spray having a viscosity of from about 100 cP to about 2,500 cP.

77. (canceled)
